(19) United States
(12) Patent Application Publication
(10) Pub. No.: US 2026/0263431 A1
(43) Pub. Date: Sep. 10, 2026

(54) ARYL HYDROCARBON RECEPTOR AGONIST PRODRUGS AND METHODS OF USE THEREOF

(71) Applicants: Allianthera (Suzhou) Biopharmaceutical Co., Ltd., Jiangsu (CN); Allianthera Boston Inc., Natick, MA (US)

(72) Inventors: Jinshan Chen, Madison, CT (US); Jiamin Gu, Jiangsu (CN)

(21) Appl. No.: 18/994,939

(22) PCT Filed: Jul. 21, 2023
(86) PCT No.: PCT/US23/28312

§ 371 Date: Jan. 15, 2025

(30) Foreign Application Priority Data
Jul. 21, 2022 (WO) PCT/CN2022/107004

(51) Int. Cl. A61K 31/44; A61K 31/12; A61K 31/192; A61K 31/196; A61K 31/255; A61K 31/405; A61K 31/407; A61K 31/421; A61K 31/472; A61K 31/505; A61K 31/5415; A61K 31/635; A61K 31/675; C07C 305/24; C07D 213/65; C07D 217/20; C07D 239/42; C07F 9/60; C07F 9/62

(52) U.S. Cl. CPC A61K 31/44; A61K 31/12; A61K 31/192; A61K 31/196; A61K 31/255; A61K 31/405; A61K 31/407; A61K 31/421; A61K 31/472; A61K 31/505; A61K 31/5415; A61K 31/635; A61K 31/675; C07C 305/24; C07D 213/65; C07D 217/20; C07D 239/42; C07F 9/60; C07F 9/62

(57) ABSTRACT

Provided herein are compounds which can act agonists of the aryl hydrocarbon receptor (AHR). Further disclosed herein are methods for treating inflammatory and autoimmune diseases and disorders, such as diseases or disorders of the gastrointestinal tract, skin, lung, central nervous system, pancreas, eye, bones, bone joints, neuroinflammatory diseases, or neurodegenerative diseases.

### FIELD

This disclosure relates to prodrug analogs of compounds that act as aryl hydrocarbon receptor (AHR) agonists, pharmaceutical formulations thereof, and methods of using the compounds to treat diseases and disorders, such as inflammatory and autoimmune diseases and disorders.

### BACKGROUND

The aryl hydrocarbon receptor (AHR) is a ligand-dependent transcription factor that regulates gene expression in a variety of cells, such as epithelial and immune cells. Activation of the AHR can lead to changes of the inflammation cytokine profile and antioxidant response in the cellular environment and can facilitate the healing process of a diseased tissue. AHR signaling can play a key role in maintaining skin and ocular homeostasis by regulating the skin immune network, keratinocyte differentiation, skin barrier function and pigmentation, retinal epithelial cell, rod photoreceptors, choroidal endothelial cells, choroidal neovascular formation, and response to oxidative stress. Cells found in the skin and eye that express AHR, includes keratinocytes, sebocytes, fibroblasts, melanocytes, endothelial cells, Langerhans cells, choroidal endothelial cells, retinal pigment epithelial cells, rod photoreceptors and immune cells present in the skin and eye. Thus, modulation of the AHR signal transduction pathway is implicated in the pathology of various diseases and disorders of the skin and eye.

AHR signaling also is known to regulate the composition and function of different cell types in the gastrointestinal tract, and therefore, has a prominent role in maintaining the balance between health and disease. AHR is involved in several physiological processes, including regulation of homeostasis and immunity at epithelial barriers such as the one formed by intestinal epithelial cells (IECs). Potent immune responses in the body occur in the gut and, as such, there is considerable interest in elucidating the molecular mechanism(s) underlying the role of AHR in cells in the intestinal mucosa—including IECs and various immune cells, such as B cells, T cell receptor γδ T cells (TCRγδ), T helper 17 cells (Th17), regulatory T cells (Treg), type 1 regulatory T cells (Tr1), innate lymphoid cells (ILC), macrophages (MQ), intraepithelial lymphocytes (IEL), dendritic cells (DC), and neutrophils. Accordingly, aberrant AHR activity has been implicated in several intestinal pathologies, such as intestinal inflammation, infection and cancer.

AHR signaling also is known to play a primary role in bone remodeling by altering the interplay between bone-forming osteoblasts and bone-resorbing osteoclasts. The overall effect of AHR activation in osteoblasts is suppressed cell differentiation, and AHR agonism has dose-dependent effects on osteoblasts in which hyperactivation and hypoactivation, respectively, inhibit and promote bone formation. The AHR pathway has been implicated in both stimulation and impairment of osteoclast differentiation. Accordingly, the AHR pathway is an attractive target for the treatment of various human diseases in which osteoblasts and osteoclasts are implicated in pathogenesis, including bone destructive diseases such as osteoporosis and cancer.

Thus, compounds that modulate the AHR pathway can used in the treatment of disorders and diseases of the gastrointestinal tract, skin, lung, central nervous system, pancreas, eye, bones, bone joints, neuroinflammatory diseases, and neurodegenerative diseases.

### SUMMARY

Provided herein are compounds having a structure of Formula (I):

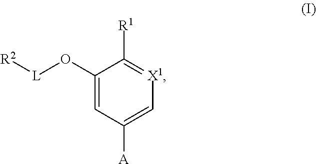

wherein: A is —Y¹—Z¹ or a fused bicyclic heteroaryl group having 8-10 total ring atoms and 1, 2, or 3 heteroatoms selected from N, O, or S, and the heteroaryl is optionally substituted with 1, 2, or 3 R^{a}; Y¹ is

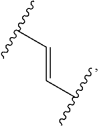 [SMILES: */C=C/*]

O, S, SO, or SO₂; Z¹ is phenyl or a 5- to 6-membered heteroaryl having 1, 2, or 3 heteroatoms selected from N, O, and S, and the phenyl or heteroaryl is optionally substituted with 1, 2, or 3 R^{a}; X¹ is N, CR³ or C—O-L-R²; R¹ is C₁₋₅alkyl, C₁₋₅haloalkyl, C₁₋₅hydroxyalkyl, OC₁₋₅alkyl, C₃₋₆cycloalkyl, N(R^{N})₂, C(═O)C₁₋₅alkyl, or C(═CH)C₁₋₄alkyl; each R² independently is SO₂—R⁴, PO(R⁴)₂, CO₂H, N(R^{N})₂, N(H)—C₂₋₆alkylene-N(R^{N})₂, N(H)—C₂₋₆polyoxyalkylene-N(R^{N})₂, NHCH(R⁵)CO₂H, NH—C₁₋₆alkylene-CH(NH₂)CO₂H, a saccharide, or NH-phenyl wherein the phenyl is optionally substituted with 1, 2, or 3 R^{b}; R³ is halo, C₁₋₆alkoxy, OH, or N(R^{N})₂; each L independently is C(O), C(O)—Wₙ—C(O), C(O)—Wₙ, or C(O)NH—Wₙ, or when R² is SO₂—R⁴, PO(R⁴)₂, or a saccharide, L can be a bond; each W independently is CH₂, C₂₋₃alkylene-O, or O—C₂₋₃alkylene; n is 1, 2, 3, 4, 5, or 6; each R⁴ independently is OH or NH₂; each R⁵ independently is H, C₁₋₆alkyl, or C₁₋₆alkylene-R⁶; R⁶ is CO₂H, CO₂C₁₋₃alkyl, CON(R^{N})₂, OH, OCH₃, N(R^{N})₂, NHC(NH)NH₂, heteroaryl comprising 5 or 6 total ring atoms and 1, 2, or 3 heteroatoms selected from N, O, and S, or phenyl, wherein the heteroaryl or phenyl is optionally substituted with 1, 2, 3, 4, or 5 C₀₋₂alkylene-R^{b} substituents; each R^{a} independently is halo, C₁₋₆alkyl, CO₂H, CO₂C₁₋₃alkyl, CON(R^{N})₂, OH, OC₁₋₃alkyl, or N(R^{N})₂; each R^{b} independently is CO₂H, CO₂C₁₋₃alkyl, or OH; and each R^{N} independently is H or C₁₋₃alkyl; or a pharmaceutically acceptable salt thereof, provided that when Y¹ is

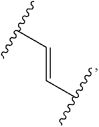 [SMILES: */C=C/*]

Z¹ is unsubstituted phenyl, and R¹ is isopropyl, then X¹ is N or CR³, and R³ is halo, C₁₋₆alkoxy, or N(R^{N})₂.

Further provided herein are pharmaceutical compositions comprising the compounds as disclosed herein. Also provided are methods of treating or preventing a disease or disorder associated with aberrant aryl hydrocarbon receptor (AHR) activity in a subject, comprising administering to the subject a therapeutically effective amount of a compound as disclosed herein.

Further aspects and advantages will be apparent to those of ordinary skill in the art from a review of the following detailed description, taken in conjunction with the drawings. While the compounds and methods disclosed herein are susceptible of cases in various forms, the description hereafter includes specific cases with the understanding that the disclosure is illustrative, and is not intended to limit the invention to the specific cases described herein.

### DETAILED DESCRIPTION

There is a need for novel aryl hydrocarbon receptor (AHR) agonists to provide new and effective therapies for diseases and disorders, such as inflammatory and autoimmune diseases and disorders. Provided herein are compounds that can act as AHR agonists, such as compounds of Formula (I):

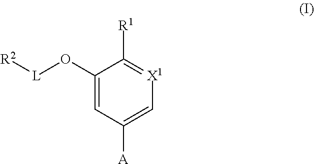

wherein A, X¹, R¹, R², and L are as described herein.

### Compounds of the Disclosure

Disclosed herein are compounds having a structure of Formula (I):

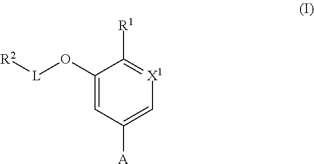

wherein: A is —Y¹—Z¹ or a fused bicyclic heteroaryl group having 8-10 total ring atoms and 1, 2, or 3 heteroatoms selected from N, O, or S, and the heteroaryl is optionally substituted with 1, 2, or 3 R^{a}; Y¹ is

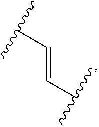 [SMILES: */C=C/*]

O, S, SO, or SO₂; Z¹ is phenyl or a 5- to 6-membered heteroaryl having 1, 2, or 3 heteroatoms selected from N, O, and S, and the phenyl or heteroaryl is optionally substituted with 1, 2, or 3 R^{a}; X¹ is N, CR³ or C—O-L-R²; R¹ is C₁₋₅alkyl, C₁₋₅ haloalkyl, C₁₋₅hydroxyalkyl, OC₁₋₅alkyl, C₃₋₆cycloalkyl, N(R^{N})₂, C(═O)C₁₋₄alkyl, or C(═CH)C₁₋₄alkyl; each R² independently is SO₂—R⁴, PO(R⁴)₂, CO₂H, N(R^{N})₂, N(H)—C₂₋₆alkylene-N(R^{N})₂, N(H)—C₂₋₆polyoxyalkylene-N(R^{N})₂, NHCH(R⁵)CO₂H, NH—C₁₋₆alkylene-CH(NH₂)CO₂H, a saccharide, or NH-phenyl wherein the phenyl is optionally substituted with 1, 2, or 3 R^{b}; R³ is halo, C₁₋₆alkoxy, OH, or N(R^{N})₂; each L independently is C(O), C(O)—Wₙ—C(O), C(O)—Wₙ, or C(O)NH—Wₙ, or when R² is SO₂—R⁴, PO(R⁴)₂, or a saccharide, L can be a bond; each W independently is CH₂, C₂₋₃alkylene-O, or O—C₂₋₃alkylene; n is 1, 2, 3, 4, 5, or 6; each R⁴ independently is OH or NH₂; each R⁵ independently is H, C₁₋₆alkyl, or C₁₋₆alkylene-R⁶; R⁶ is CO₂H, CO₂C₁₋₃alkyl, CON(R^{N})₂, OH, OCH₃, N(R^{N})₂, NHC(NH)NH₂, heteroaryl comprising 5 or 6 total ring atoms and 1, 2, or 3 heteroatoms selected from N, O, and S, or phenyl, wherein the heteroaryl or phenyl is optionally substituted with 1, 2, 3, 4, or 5 C₀₋₂alkylene-R^{b} substituents; each R^{a} independently is halo, C₁₋₆alkyl, CO₂H, CO₂C₁₋₃alkyl, CON(R^{N})₂, OH, OC₁₋₃alkyl, or N(R^{N})₂; each R^{b} independently is CO₂H, CO₂C₁₋₃alkyl, or OH; and each R^{N} independently is H or C₁₋₃alkyl; or a pharmaceutically acceptable salt thereof, provided that when Y¹ is

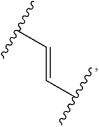 [SMILES: */C=C/*]

Z¹ is unsubstituted phenyl, and R¹ is isopropyl, then X¹ is N or CR³, and R³ is halo, C₁₋₆alkoxy, or N(R^{N})₂.

In compounds of Formula (I), A can be —Y¹—Z¹ or a fused bicyclic heteroaryl group having 8-10 total ring atoms and 1, 2, or 3 heteroatoms selected from N, O, or S, and the heteroaryl is optionally substituted with 1, 2, or 3 R^{a}. In various cases, A is —Y¹—Z¹. In compounds of Formula (I), Y¹ can be

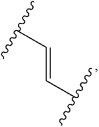 [SMILES: */C=C/*]

O, S, SO, or SO₂. In various cases, Y¹ is

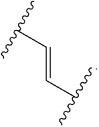 [SMILES: */C=C/*]

In some cases, Y¹ is S, SO, or SO₂. In compounds of Formula (I), Z¹ can be phenyl or a 5- to 6-membered heteroaryl having 1, 2, or 3 heteroatoms selected from N, O, and S, and the phenyl or heteroaryl is optionally substituted with 1, 2, or 3 R^{a}. In various cases, Z¹ is phenyl. In some cases, Z¹ is a 5- to 6-membered heteroaryl. In some cases, Z¹ is pyridine, pyrazole, imidazole, or pyrimidine. In some cases, Z¹ is unsubstituted. In some cases, Z¹ is substituted with 1 or 2 R^{a}; wherein each R^{a} is independently halo, C₁₋₃alkyl, or NH₂. In various cases, at least one R^{a} of substituted Z¹ is halo. In various cases, at least one R^{a} of substituted Z¹ is OH. In some cases, at least one R^{a} of substituted Z¹ is CO₂H. In some cases, at least one R^{a} of substituted Z¹ is CO₂C₁₋₃alkyl. In some cases, at least one R^{a} of substituted Z¹ is CON(R^{N})₂. In some cases, at least one R^{a} of substituted Z¹ is OC₁₋₃alkyl. In some cases, at least one R^{a} of substituted Z¹ is N(R^{N})₂.

In various cases, A is a fused bicyclic heteroaryl. In some cases, A is a 10-membered heteroaryl. In some cases, A is isoquinoline, quinoline, or quinazoline. In some cases, A is

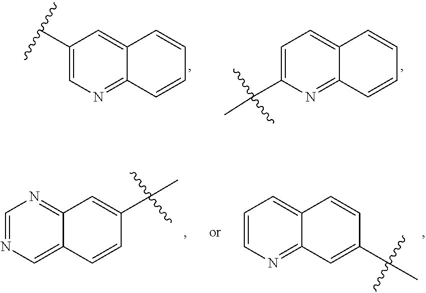 [SMILES: *c1cnc2ccccc2c1 ; *c1ccc2ccccc2n1 ; *c1ccc2cncnc2c1 ; *c1ccc2cccnc2c1]

or if R² is SO₂—R⁴ or PO(R⁴)₂, A is

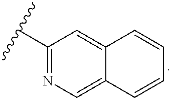 [SMILES: *c1cc2ccccc2cn1]

In some cases, A is unsubstituted. In various cases, A is substituted with 1 or 2 R^{a}; wherein each R^{a} is independently halo, C₁₋₃alkyl, or NH₂. In various cases, at least one R^{a} of substituted A is halo. In various cases, at least one R^{a} of substituted A is OH. In some cases, at least one R^{a} of substituted A is CO₂H. In some cases, at least one R^{a} of substituted A is CO₂C₁₋₃alkyl. In some cases, at least one R^{a} of substituted A is CON(R^{N})₂. In some cases, at least one R^{a} of substituted A is OC₁₋₃alkyl. In some cases, at least one R^{a} of substituted A is N(R^{N})₂.

In some cases, A or Z¹ are substituted with 1, 2, or 3 R^{a} groups. In some cases, A or Z¹ are substituted with 1 or 2 R^{a} groups. In compounds of Formula (I), each R^{a} can independently be halo, C₁₋₆alkyl, CO₂H, CO₂C₁₋₃alkyl, CON(R^{N})₂, OH, OC₁₋₃alkyl, or N(R^{N})₂. In various cases, at least one R^{a} is halo. In various cases, at least one R^{a} is OH. In some cases, at least one R^{a} is CO₂H. In some cases, at least one R^{a} is CO₂C₁₋₃alkyl. In some cases, at least one R^{a} is CON(R^{N})₂. In some cases, at least one R^{a} is OC₁₋₃alkyl. In some cases, at least one R^{a} is N(R^{N})₂.

In compounds of Formula (I), X¹ can be N, CR³ or C—O-L-R². In various cases, X¹ is N. In various cases, X¹ is CR³. In compounds of Formula (I), R³ can be halo, OH, or N(R^{N})₂. In some cases, R³ is F or OH. In some cases, R³ is N(R^{N})₂. In various cases, X¹ is C—O-L-R². In various cases, X¹ is C—O—R². In various cases, X¹ is C—OSO₂—R⁴ or C—OPO(R⁴)₂.

In compounds of Formula (I), R¹ can be C₁₋₅alkyl, C₁₋₅haloalkyl, C₁₋₅hydroxyalkyl, OC₁₋₅alkyl, C₃₋₆cycloalkyl, N(R^{N})₂, C(═O)C₁₋₄alkyl, or C(═CH)C₁₋₄alkyl. In various cases, R¹ is C₁₋₅alkyl, C₁₋₅haloalkyl, or C₁₋₅ hydroxyalkyl. In some cases, R¹ is C₁₋₃alkyl. In various cases, R¹ is C₃alkyl. In some cases, R¹ is OC₁₋₅alkyl, N(R^{N})₂, or C(═O)C₁₋₄alkyl.

In compounds of Formula (I), each L independently can be C(O), C(O)—Wₙ—C(O), C(O)—Wₙ, or C(O)NH—Wₙ, or when R² is SO₂—R⁴, PO(R⁴)₂, or a saccharide, L can be a bond. In various cases, L is a bond. In some cases, each L is independently C(O), C(O)—W—C(O), C(O)—Wₙ, or C(O)NH—Wₙ. In compounds of Formula (I), each W independently can be CH₂, C₂₋₃alkylene-O, or O—C₂₋₃alkylene. In various cases, W is CH₂ or C₂₋₃ alkylene-O. In compounds of Formula (I), n can be 1, 2, 3, 4, 5, or 6. In various cases, n is 1 or 2.

In compounds of Formula (I), each R² independently can be SO₂—R⁴, PO(R⁴)₂, CO₂H, N(R^{N})₂, N(H)—C₂₋₆alkylene-N(R^{N})₂, N(H)—C₂₋₆polyoxyalkylene-N(R^{N})₂, NHCH(R⁵)CO₂H, NH—C₁₋₆alkylene-CH(NH₂)CO₂H, a saccharide, or NH-phenyl wherein the phenyl is optionally substituted with 1, 2, or 3 R^{b}; wherein each R^{b} can independently be CO₂H, CO₂C₁₋₃alkyl, or OH. In various cases, at least one R^{b} is CO₂H or OH. In compounds of Formula (I), each R^{N} can independently be H or C₁₋₃alkyl. In various cases, R² is SO₂—R⁴ or PO(R⁴)₂. In various cases, R² is SO₂H or PO(OH)₂. In some cases, R² is N(H)—C₂₋₆alkylene-N(R^{N})₂, N(H)—C₂₋₆polyoxyalkylene-N(R^{N})₂, NHCH(R⁵)CO₂H, or NH—C₁₋₆alkylene-CH(NH₂)CO₂H. In some cases, R² is N(H)—C₂₋₆alkylene-N(R^{N})₂ or NH—C₁₋₆alkylene-CH(NH₂)CO₂H. In some cases, R² is NHCH(R⁵)CO₂H. In various cases, at least one R² is NH-phenyl.

In compounds of Formula (I), each R⁴ can independently be OH or NH₂. In various cases, at least one R⁴ is OH. In some cases, at least one R⁴ is NH₂.

In compounds of Formula (I), each R⁵ independently is H, C₁₋₅alkyl, or C₁₋₆alkylene-R⁶. In various cases, each R⁵ is independently C₁₋₅alkyl or C₁₋₆alkylene-R⁶.

In compounds of Formula (I), each R⁵ can independently be CO₂H, CO₂C₁₋₃alkyl, CON(R^{N})₂, OH, OCH₃, N(R^{N})₂, NHC(NH)NH₂, heteroaryl comprising 5 or 6 total ring atoms and 1, 2, or 3 heteroatoms selected from N, O, and S, or phenyl, wherein the heteroaryl or phenyl is optionally substituted with 1, 2, 3, 4, or 5 C₀₋₂alkylene-R^{b} substituents. In various cases, R⁶ is CO₂H, CO₂CH₃, CONH₂, OH, OCH₃, NH₂, NHC(NH)NH₂, imidazolyl, phenyl, or 4-hydroxy-phenyl.

In various cases, at least one R² is NH-phenyl; wherein the phenyl is substituted with 1 or 2 R^{b} groups. In some cases, each R² and/or R⁶ is substituted with 1 to 5 R^{b} groups. In compounds of Formula (I), each R^{b} can independently be CO₂H, CO₂C₁₋₃alkyl, or OH. In various cases, at least one R^{b} is CO₂H or OH. In compounds of Formula (I), each R^{N} can independently be H or C₁₋₃alkyl. In various cases, each R^{N} is H. In some cases, at least one R^{N} is CH₃. In some cases, at least one R^{N} is CH₂CH₃.

In some cases, the compound of Formula (I) is (E)-2-isopropyl-5-styrylpyridin-3-yl hydrogen sulfate, (E)-2-isopropyl-5-styrylpyridin-3-yl dihydrogen phosphate, 3-hydroxy-2-isopropyl-5-(isoquinolin-3-yl)phenyl hydrogen sulfate, 3-hydroxy-2-isopropyl-5-(isoquinolin-3-yl)phenyl dihydrogen phosphate, 3-hydroxy-2-isopropyl-5-(quinolin-3-yl)phenyl hydrogen sulfate, 3-hydroxy-2-isopropyl-5-(quinolin-3-yl)phenyl dihydrogen phosphate, 2-isopropyl-5-(quinolin-3-yl)-1,3-phenylene bis(hydrogen sulfate), 2-isopropyl-5-(quinolin-3-yl)-1,3-phenylene bis(dihydrogen phosphate), (E)-5-(2-fluorostyryl)-2-isopropylpyridin-3-yl hydrogen sulfate, (E)-5-(2-fluorostyryl)-2-isopropylpyridin-3-yl dihydrogen phosphate, (E)-5-(3-fluorostyryl)-2-isopropylpyridin-3-yl hydrogen sulfate, (E)-5-(3-fluorostyryl)-2-isopropylpyridin-3-yl dihydrogen phosphate, (E)-5-(4-fluorostyryl)-2-isopropylpyridin-3-yl hydrogen sulfate, (E)-5-(4-fluorostyryl)-2-isopropylpyridin-3-yl dihydrogen phosphate, 5-(2-amino-5-fluoroquinazolin-7-yl)-3-hydroxy-2-isopropylphenyl hydrogen sulfate, 5-(2-amino-5-fluoroquinazolin-7-yl)-3-hydroxy-2-isopropylphenyl dihydrogen phosphate, 5-(2-amino-5-fluoroquinazolin-7-yl)-2-isopropyl-1,3-phenylene bis(hydrogen sulfate); 5-(2-amino-5-fluoroquinazolin-7-yl)-2-isopropyl-1,3-phenylene bis(dihydrogen phosphate); 5-(6-fluoroquinolin-2-yl)-3-hydroxy-2-isopropylphenyl hydrogen sulfate, 5-(6-fluoroquinolin-2-yl)-3-hydroxy-2-isopropylphenyl dihydrogen phosphate, 5-(6-fluoroquinolin-2-yl)-2-isopropyl-1,3-phenylene bis(hydrogen sulfate), 5-(6-fluoroquinolin-2-yl)-2-isopropyl-1,3-phenylene bis(dihydrogen phosphate), (E)-3-fluoro-2-propyl-5-styrylphenyl hydrogen sulfate, (E)-3-fluoro-2-propyl-5-styrylphenyl dihydrogen phosphate, (E)-3-fluoro-2-isopropyl-5-styrylphenyl hydrogen sulfate, (E)-3-fluoro-2-isopropyl-5-styrylphenyl dihydrogen phosphate, 3-fluoro-2-isopropyl-5-(4-methylquinolin-7-yl)phenyl hydrogen sulfate, or 3-fluoro-2-isopropyl-5-(4-methylquinolin-7-yl)phenyl dihydrogen phosphate, or a pharmaceutically acceptable salt of any of the foregoing.

In some cases, the compound of Formula (I) is 2-isopropyl-5-(quinolin-3-yl)-1,3-phenylene bis(dihydrogen phosphate), 2-isopropyl-5-(quinolin-3-yl)-1,3-phenylene bis(hydrogen sulfate), (E)-5-(2-fluorostyryl)-2-isopropylpyridin-3-yl hydrogen sulfate, (E)-5-(2-fluorostyryl)-2-isopropylpyridin-3-yl dihydrogen phosphate, (E)-3-fluoro-2-propyl-5-styrylphenyl hydrogen sulfate, (E)-3-fluoro-2-propyl-5-styrylphenyl dihydrogen phosphate, (E)-3-fluoro-2-isopropyl-5-styrylphenyl hydrogen sulfate, (E)-3-fluoro-2-isopropyl-5-styrylphenyl dihydrogen phosphate, 3-fluoro-2-isopropyl-5-(4-methylquinolin-7-yl)phenyl hydrogen sulfate, or 3-fluoro-2-isopropyl-5-(4-methylquinolin-7-yl)phenyl dihydrogen phosphate, or a pharmaceutically acceptable salt of any of the foregoing.

In some cases, the compound of Formula (I) is

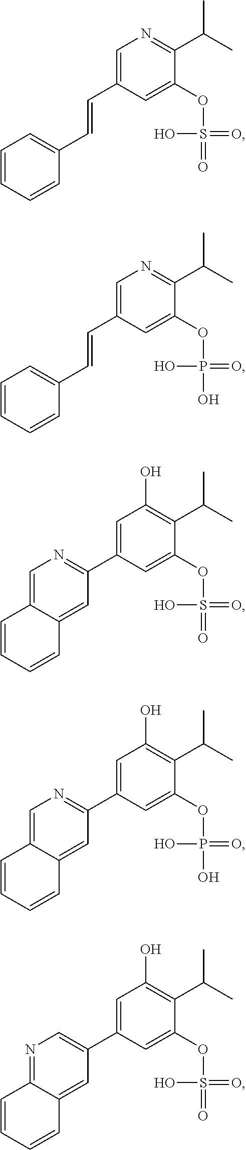 [SMILES: CC(C)c1ncc(/C=C/c2ccccc2)cc1OS(=O)(=O)O ; CC(C)c1ncc(/C=C/c2ccccc2)cc1OP(=O)(O)O ; CC(C)c1c(O)cc(-c2cc3ccccc3cn2)cc1OS(=O)(=O)O ; CC(C)c1c(O)cc(-c2cc3ccccc3cn2)cc1OP(=O)(O)O ; CC(C)c1c(O)cc(-c2cnc3ccccc3c2)cc1OS(=O)(=O)O]

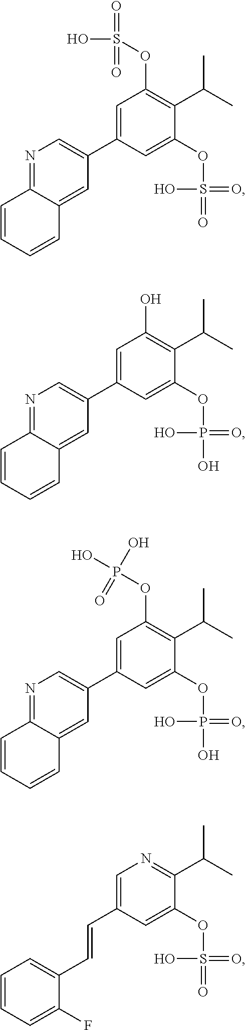 [SMILES: CC(C)c1c(OS(=O)(=O)O)cc(-c2cnc3ccccc3c2)cc1OS(=O)(=O)O ; CC(C)c1c(O)cc(-c2cnc3ccccc3c2)cc1OP(=O)(O)O ; CC(C)c1c(OP(=O)(O)O)cc(-c2cnc3ccccc3c2)cc1OP(=O)(O)O ; CC(C)c1ncc(/C=C/c2ccccc2F)cc1OS(=O)(=O)O]

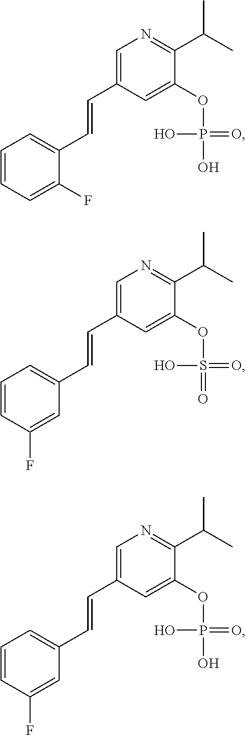 [SMILES: CC(C)c1ncc(/C=C/c2ccccc2F)cc1OP(=O)(O)O ; CC(C)c1ncc(/C=C/c2cccc(F)c2)cc1OS(=O)(=O)O ; CC(C)c1ncc(/C=C/c2cccc(F)c2)cc1OP(=O)(O)O]

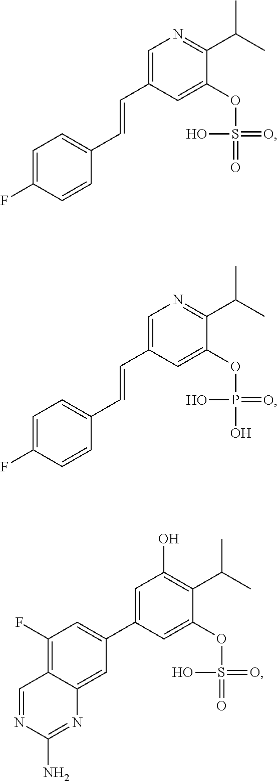 [SMILES: CC(C)c1ncc(/C=C/c2ccc(F)cc2)cc1OS(=O)(=O)O ; CC(C)c1ncc(/C=C/c2ccc(F)cc2)cc1OP(=O)(O)O ; CC(C)c1c(O)cc(-c2cc(F)c3cnc(N)nc3c2)cc1OS(=O)(=O)O]

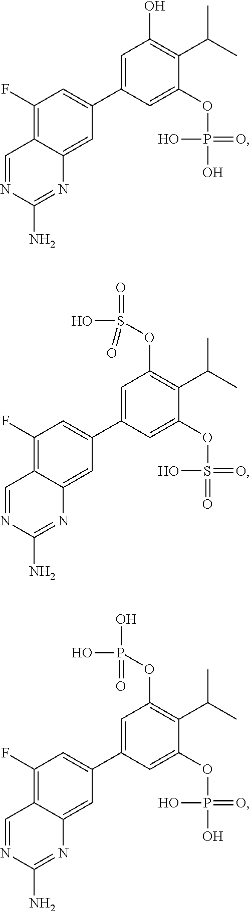 [SMILES: CC(C)c1c(O)cc(-c2cc(F)c3cnc(N)nc3c2)cc1OP(=O)(O)O ; CC(C)c1c(OS(=O)(=O)O)cc(-c2cc(F)c3cnc(N)nc3c2)cc1OS(=O)(=O)O ; CC(C)c1c(OP(=O)(O)O)cc(-c2cc(F)c3cnc(N)nc3c2)cc1OP(=O)(O)O]

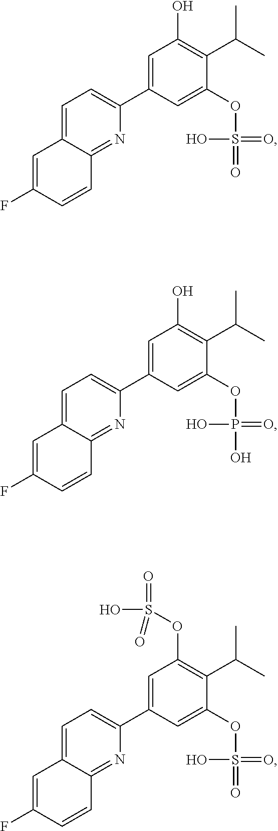 [SMILES: CC(C)c1c(O)cc(-c2ccc3cc(F)ccc3n2)cc1OS(=O)(=O)O ; CC(C)c1c(O)cc(-c2ccc3cc(F)ccc3n2)cc1OP(=O)(O)O ; CC(C)c1c(OS(=O)(=O)O)cc(-c2ccc3cc(F)ccc3n2)cc1OS(=O)(=O)O]

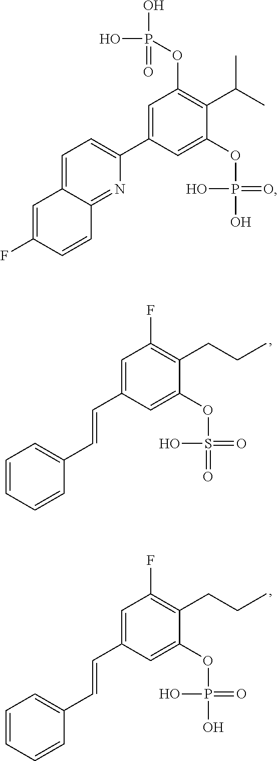 [SMILES: CC(C)c1c(OP(=O)(O)O)cc(-c2ccc3cc(F)ccc3n2)cc1OP(=O)(O)O ; CCCc1c(F)cc(/C=C/c2ccccc2)cc1OS(=O)(=O)O ; CCCc1c(F)cc(/C=C/c2ccccc2)cc1OP(=O)(O)O]

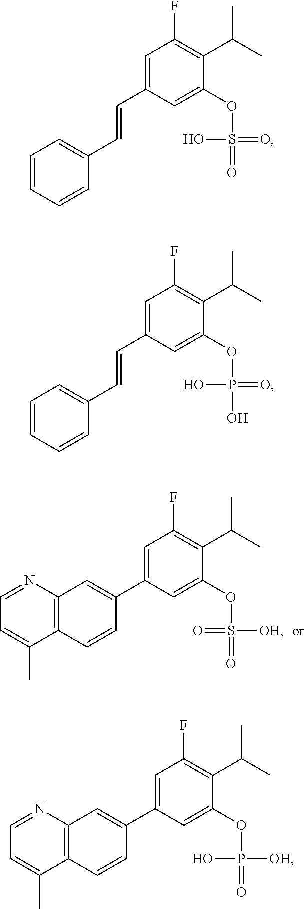 [SMILES: CC(C)c1c(F)cc(/C=C/c2ccccc2)cc1OS(=O)(=O)O ; CC(C)c1c(F)cc(/C=C/c2ccccc2)cc1OP(=O)(O)O ; Cc1ccnc2cc(-c3cc(F)c(C(C)C)c(OS(=O)(=O)O)c3)ccc12 ; Cc1ccnc2cc(-c3cc(F)c(C(C)C)c(OP(=O)(O)O)c3)ccc12]

or a pharmaceutically acceptable salt thereof. In some cases, the compound of Formula (I) is

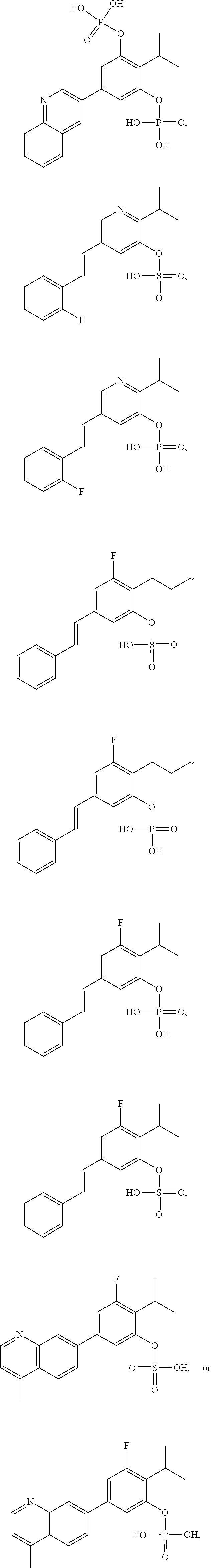

or a pharmaceutically acceptable salt thereof.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

The compounds disclosed herein that have a double bond can exhibit E or Z (not shown) stereochemistry at the double bond of Y¹. In some cases, the compounds of Formula (I) exhibit E stereochemistry at the double bond of Y¹. In various cases, the compounds of Formula (I) exhibit Z stereochemistry at the double bond. The compounds of Formula (I) can have any stereochemical configuration at any sp³ carbon atoms. In some cases, the compounds of the disclosure are optically pure. As used herein, “optically pure” refers to the predominant presence of one enantiomer of a compound if multiple stereochemical configurations can exist (e.g., at least 99% enantiomeric excess).

Unless otherwise indicated, all tautomeric forms of the compounds of the disclosure are within the scope of the disclosure.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

As used herein, “alkyl” refers to straight chained and branched saturated hydrocarbon groups containing one to thirty carbon atoms, for example, one to four carbon atoms (e.g., 1, 2, 3, or 4). The term Cn means the alkyl group has “n” carbon atoms. For example, C₃ alkyl refers to an alkyl group that has 3 carbon atoms. C₁₋₄alkyl refers to an alkyl group having a number of carbon atoms encompassing the entire range (i.e., 1 to 4 carbon atoms), as well as all subgroups (e.g., 1-2, 1-3, 2-3, 2-4, 1, 2, 3, and 4 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, “alkylene” refers to a bivalent saturated aliphatic radical. The term Cn means the alkylene group has “n” carbon atoms. For example, C₁₋₆alkylene refers to an alkylene group having a number of carbon atoms encompassing the entire range, as well as all subgroups, as previously described for “alkyl” groups. As used herein, “polyoxyalkylene” refers to an alkylene group with an ether linkage present on the terminal end and optionally within the alkylene chain. Non-limiting examples of C₂₋₆polyoxyalkylene include a polyethylene glycol moiety and a polypropylene glycol moiety, such as C₂polyoxyalkylene (e.g., —CH₂CH₂OCH₂CH₂O— and —CH₂CH₂CH₂OCH₂CH₂CH₂O—).

As used herein, the term “haloalkyl” refers to an alkyl group in which one or more of the hydrogen atoms are replaced by halogen. Such groups include but are not limited to, chloromethyl, fluoromethyl, difluoromethyl, trifluoromethyl, 1,1-difluoroethyl, 2-fluoroethyl, 1-chloro-2-fluoromethyl and 2-fluoroisobutyl.

As used herein, the term “hydroxyalkyl” refers to an alkyl group in which one or more of the hydrogen atoms are replaced by a hydroxyl group (OH). Such groups include but are not limited to, hydroxymethyl, hydroxyethyl, and the like.

As used herein, the term “heteroaryl” refers to a cyclic aromatic ring having heteroatoms in the ring (e.g., a monocyclic aromatic ring with 5-6 total ring atoms, or a fused bicyclic ring with 10 total ring atoms), and containing one to three heteroatoms selected from nitrogen, oxygen, and sulfur atom in the aromatic ring. Unless otherwise indicated, a heteroaryl group can be unsubstituted or substituted. Heteroaryl groups can be isolated (e.g., pyridyl) or fused to another heteroaryl group (e.g., purinyl), a cycloalkyl group (e.g., tetrahydroquinolinyl), a heterocycloalkyl group (e.g., dihydronaphthyridinyl), and/or an aryl group (e.g., benzothiazolyl, quinolyl, isoquinolinyl, or quinazolinyl). Examples of contemplated heteroaryl groups include, but are not limited to, pyrazolyl, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

As used herein, a fused bicyclic heteroaryl refers to heteroaryl group fused to another heteroaryl group and each ring can contain five or six total ring atoms and one to three heteroatoms in its aromatic ring. Examples of fused bicyclic heteroaryl groups include, but are not limited to, benzooxazolyl, benzoimidazolyl, indolyl, isoindolyl, indolizinyl, purinyl, benzofuranyl, benzimidazole, quinolinyl, isoquinolinyl, naphthridinyl, and pyridopyrazinyl.

As used herein, the term “halo” refers to a fluoro (F), chloro (Cl), bromo (Br), or iodo (I) group.

As used herein, a “substituted” functional group is a functional, group having at least one hydrogen radical that is substituted with a non-hydrogen radical (i.e., a substituent). Examples of non-hydrogen radicals (or substituents) include, but are not limited to, alkyl, cycloalkyl, alkenyl, cycloalkyl, alkynyl, ether, aryl, heteroaryl, heterocycloalkyl, hydroxyl, oxy (or oxo), alkoxyl, ester, thioester, acyl, carboxyl, cyano, nitro, amino, sulfhydryl, and halo. When a substituted alkyl group includes more than one non-hydrogen radical, the substituents can be bound to the same carbon or two or more different carbon atoms.

As used herein, the saccharide can be any saccharide known in the art, such as a monosaccharide (e.g., a simple sugar having a general formula C₆H₁₂O₆), a disaccharide (e.g., a saccharide comprising two monosaccharides linked together by a glycosidic bond), an oligosaccharide (e.g., a saccharide comprising 3-10 monosaccharides linked together by glycosidic bonds), a polysaccharide (e.g., a saccharide comprising seven or more monosaccharides linked together by glycosidic bonds), or an amino saccharide (e.g., a saccharide in which a hydroxyl group has been replace with an amino group). As used herein, a glycosidic bond is bond between a hemiacetal or hemiketal group of a saccharide (or a molecule derived from a saccharide) and the hydroxyl group of a second saccharide. Each glycosidic bond can independently be an α-glycosidic bond (e.g., the carbon atoms of each partner of the glycosidic bond have the same stereochemistry) or a β-glycosidic bond (e.g., the carbon atoms of each partner of the glycosidic bond have different stereochemistry). In some cases, the oligosaccharide comprises α-glycosidic bonds. In some embodiments, the saccharide is a monosaccharide, a disaccharide, an oligosaccharide, or an aminosaccharide. Contemplated monosaccharides include, but are not limited to, glucose, fructose, galactose, ribose, and xylose. In some embodiments, the saccharide is an oligosaccharide comprising 3-10 monosaccharide units. In various embodiments, the oligosaccharide comprises α-1,4 glycosidic bonds. In some cases, the oligosaccharide is a cyclic oligosaccharide having 5-8 pyranose units. In some embodiments, the pyranose units comprise glucose. In various embodiments, the oligosaccharide comprises a cyclodextrin. In some cases, at least one —O-L-R² is

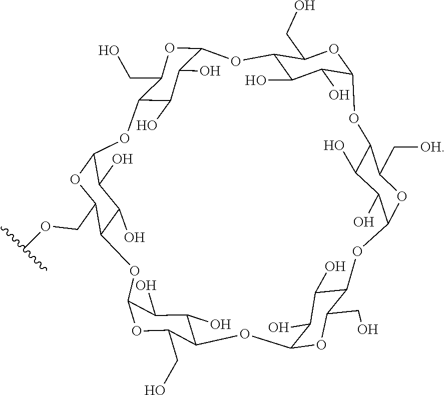 [SMILES: *OCC1OC2OC3C(CO)OC(OC4C(CO)OC(OC5C(CO)OC(OC6C(CO)OC(OC7C(CO)OC(OC1C(O)C2O)C(O)C7O)C(O)C6O)C(O)C5O)C(O)C4O)C(O)C3O]

### Pharmaceutically Acceptable Salts

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxyl group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carder, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g. orally, topically, rectally, parenterally, (for example, subcutaneous injections, intravenous, intramuscular, intrasternal, and intrathecal injection or infusion techniques), or as a buccal, inhalation, or nasal spray. The administration can be to provide a systemic effect (e.g. eneteral or parenteral). All methods that can be used by those skilled in the art to administer a pharmaceutically active agent are contemplated. In some cases, the disclosed formulations can be administered orally or topically.

Suitable oral compositions or formulations in accordance with the disclosure include without limitation tablets, troches, lozenges, aqueous or oily suspensions, dispersible powders or granules, emulsion, hard or soft capsules, syrups or elixirs. Compositions or formulations suitable for oral use may be prepared according to any method known to the art for the manufacture of pharmaceutical compositions.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions and formulations described herein may also be administered topically or transdermally, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract, e.g., can be effected in a rectal suppository formulation or in a suitable enema formulation. Dosage forms for topical or transdermal administration of a compound described herein include ointments, pastes, creams, lotions, gels, powders, solutions, sprays, inhalants, suppositories, or patches.

For topical applications, the pharmaceutical compositions may be formulated in a suitable ointment, cream, lotion, or gel, containing the active component suspended or dissolved in one or more carriers, and any needed preservatives or buffers as may be required. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carder. The unit dosage form can be for a single daily dose or one of multiple daily doses (e.g., about 1 to 4 or more times per day). When multiple daily doses are used, the unit dosage form can be the same or different for each dose.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Treatment

The compounds disclosed herein (e.g., the compounds of Formula (I)), and pharmaceutically acceptable salts thereof, can act as agonists of the aryl hydrocarbon receptor (AHR) pathway. AHR is a ligand-activated transcription factor that has been implicated in a variety of conditions, including by modulating the immune system during steady state and during infection and inflammation. The AHR pathway has been recognized for its role in the pathogenesis of inflammatory skin diseases such as atopic dermatitis, acne, psoriasis, and vitiligo; intestinal pathologies, such as inflammatory bowel disorder, necrotizing enterocolitis and other autoimmune diseases, and for colorectal cancer; in diseases and disorders of the bones and joints, including rheumatoid arthritis; diseases of the eye, such as age-related macular degeneration; and diseases of the lung, such as lung fibrosis, chronic obstructive pulmonary disease. The AHR pathway also has been recognized for its role in the pathogenesis of diseases and disorders including neuroinflammatory diseases and disorders, neurodegenerative diseases and disorders, cancer, diabetes, viral, and bacterial infections.

Thus, the disclosure provides a method of modulating the aryl hydrocarbon receptor (AHR) in a cell comprising contacting the cell with a therapeutically effective amount of a compound or salt disclosed herein (e.g., the compounds of Formula (I) and pharmaceutically acceptable salts of any of the foregoing) or a formulation thereof, in an amount effective to modulate the AHR. In some cases, the contacting occurs in vitro. In some cases, the contacting occurs in vivo. In some cases, the contacting comprises administering to a subject in need thereof. As used herein, the terms “patient” and “subject” may be used interchangeably and mean animals, such as dogs, cats, cows, horses, and sheep (i.e., non-human animals) and humans. Particular patients are mammals (e.g., humans). In some cases, the subject suffers from an inflammatory disease or disorder. In some cases, the subject suffers from a disease or disorder of the gastrointestinal tract, skin, lung, eye, or bone joints. In some cases, the subject suffers from cancer or a viral infection.

Another aspect of the disclosure provides a method of treating a disease or disorder in a subject, comprising administering to the subject a therapeutically effective amount of a compound or salt disclosed herein (such as a compound of Formula (I)), or a formulation thereof. In some cases, the terms “treating”, “treat” or “treatment” and the like can include preventative (e.g., prophylactic) and palliative treatment. In some cases, the disease or disorder is an inflammatory disease or disorder. In some cases, the disease or disorder is a disease or disorder of the gastrointestinal tract, skin, lung, central nervous system (“CNS”), pancreas, eye, bones, or bone joints, a neuroinflammatory disease, or a neurodegenerative disease. In some cases, the disease or disorder is diabetes, cancer, a viral infection, or a bacterial infection.

In some cases, the disease or disorder of the gastrointestinal tract is colitis, inflammatory bowel disease, Crohn's disease, celiac disease, necrotizing enterocolitis, irritable bowel syndrome, chronic idiopathic constipation, traveler's diarrhea, or colorectal cancer. In some cases, the disease or disorder of the skin is atopic dermatitis, acne, psoriasis, or vitiligo. In some cases, the disease or disorder of the eye is abnormal eye movements, inflammatory ocular disease, autoimmune ocular disease, hereditary ocular disease, degenerative ocular disease, vascularization ocular disease, dry and wet age-related macular degeneration (“AMD”), Uveitis, retinitis pigmentosa (“RP”), primary open-angle glaucoma (“POAG”), primary congenital glaucoma, Behcet's disease, or Leber congenital amaurosis (“LCA”). In some cases, the disease or disorder of the lung is lung fibrosis, asthma, or chronic obstructive pulmonary disease. In some cases, the disease or disorder of the bone or bone joints is osteoporosis, rheumatoid arthritis, or bone cancer. In some cases, the disease or disorder of the central nervous system is Alzheimer's Disease and other memory disorders, Amyotrophic Lateral Sclerosis (ALS, Lou Gehrig's disease, or motor neuron disease). In some cases, the disease or disorder of the pancreas is type 1 diabetes. In some cases, the autoimmune disease or disorder is Hidradenitis Suppurativa, Systemic Sclerosis, Lupus Nephritis, Ankylosing Spondylitis, Non-Radiographic Axial Spondyloarthritis (NR-Ax-SpA), Rheumatoid Arthritis, Juvenile Idiopathic arthritis (JIA), Crohn's Disease, Ulcerative Colitis, Systemic Lupus Erythematosus, Atopic Dermatitis, Chronic Obstructive Pulmonary Disease, Severe Asthma, Psoriasis, Sjogren's Disease, Chronic Spontaneous Urticaria, Cutaneous Lupus Erythematosus, Still's Disease, Idiopathic Pulmonary Fibrosis, Dermatomyositis, Sarcoidosis, Psoriatic Arthritis, Pustular Psoriasis, Osteoarthritis, Alopecia Areata, Giant Cell Arteritis (GCA), ANCA Vasculitis, Vasculitis, Eosinophilic Esophagitis, Acute Respiratory Distress Syndrome, Severe Acne or Lichen Planus. In some cases, the neuroinflammatory disease or disorder is Multiple Sclerosis, Rasmussen's encephalitis, acute disseminated encephalomyelitis (ADEM), Autoimmune encephalitis, transverse myelitis (TM), or anti-NMDA receptor encephalitis. In some cases, the neurodegenerative disease or disorder is Uveitis, optic neuritis, neuromyelitis optica spectrum disorder (NMOSD), or Age-related Macular Degeneration (AMD).

In some cases, the disease or disorder is diabetes (e.g., type 1 diabetes or type 2 diabetes). In some cases, the disease or disorder is cancer. Contemplated cancers that can be treated using the compounds and methods described herein include, but are not limited to, a hematological cancer, a lymphoma, a myeloma, a leukemia, a neurological cancer, skin cancer, breast cancer, a prostate cancer, a cancer of the respiratory tract, a cancer of the reproductive organs, a cancer of the digestive tract, a colorectal cancer, lung cancer, head and neck cancer, a gastrointestinal cancer, a liver cancer, a pancreatic cancer, a genitourinary cancer, a bone cancer, renal cancer, and a vascular cancer.

In some cases, the disease or disorder is a viral infection. In some cases, the compounds of the disclosure such as the compounds of Formula (I) and pharmaceutically acceptable salts of any of the foregoing, or a composition thereof are useful for the treatment of a viral infection. In some cases, the viral infection to be treated using the compounds and methods described herein include, but are not limited to, coronavirus infections (e.g., SARS, MERS, and COVID-19, such as SARS-CoV infection, MERS-CoV infection, and SARS-CoV-2 infection, respectively) and flavivirus infections (e.g., West Nile virus infection, dengue virus infection, tick-borne encephalitis virus infection, yellow fever virus infection, Zika virus infection, and several other viruses which may cause encephalitis).

In some cases, the disease or disorder is a bacterial infection. In some cases, the compounds of the disclosure such as the compounds of Formula (I) and pharmaceutically acceptable salts of any of the foregoing, or a composition thereof are useful for the treatment of a bacterial infection. In some cases, the bacterial infection to be treated using the compounds and methods described herein include, but are not limited to, a pulmonary infection, gastrointestinal infection, skin infection, ear infection or a septicemia.

Another aspect of the disclosure provides the use of a compound disclosed herein such as a compound of Formula (I), a pharmaceutically acceptable salt thereof, or a formulation comprising a compound or salt disclosed herein in the treatment of an inflammatory disease or disorder, such as disease or disorder of the gastrointestinal tract, skin, lung, central nervous system, pancreas, eye, bones or bone joints, neuroinflammatory diseases and disorders, or neurodegenerative diseases and disorders.

Also contemplated is the use of a compound disclosed herein such as a compound of Formula (I), a pharmaceutically acceptable salt thereof, or a formulation comprising a compound or salt disclosed herein for the manufacture of a medicament in the treatment of an inflammatory disease or disorder, such as disease or disorder of the gastrointestinal tract, skin, lung, central nervous system, pancreas, eye, bones or bone joints, neuroinflammatory diseases and disorders, or neurodegenerative diseases and disorders.

Use of a compound disclosed herein such as a compound of Formula (I), a pharmaceutically acceptable salt thereof, or a formulation comprising a compound or salt disclosed herein to treat an inflammatory disease or disorder, such as disease or disorder of the gastrointestinal tract, skin, lung, central nervous system, pancreas, eye, bones or bone joints, neuroinflammatory diseases and disorders, or neurodegenerative diseases and disorders also is contemplated.

In some cases, the compound of Formula (I) as disclosed herein, or pharmaceutically acceptable salt thereof, can be administered in combination with another therapeutic agent to treat a disease or disorder, such as a disease or disorder disclosed herein. In some cases, the combination therapy can be used to treat an inflammatory disease or disorder (e.g., a disease or disorder of the gastrointestinal tract, skin, eye, lung, or bone joints). The terms “co-administration,” “administration with,” “administration in combination with,” or the like, as used herein, encompass administration of the selected therapeutic agents to a single patient, and are intended to include treatment regimens in which the agents are administered by the same or different route of administration or at the same or different time. Thus, in any of the methods disclosed herein, treatment of a disease or disorder disclosed herein, such as an inflammatory disease or disorder (e.g., a disease or disorder of the gastrointestinal tract, skin, lung, central nervous system, pancreas, eye, bones or bone joints, neuroinflammatory diseases and disorders, or neurodegenerative diseases and disorders) includes co-administration of a compound of the disclosure such as a compound of Formula (I), a pharmaceutically acceptable salt thereof, or a formulation comprising a compound or salt disclosed herein in combination with another therapeutic agent. In some cases, the other therapeutic agent is an anti-inflammatory agent. Contemplated anti-inflammatory agents use for co-administration with the compounds of the disclosure include, e.g., mesalazine, naproxen, ibuprofen, diclofenac, celecoxib, sulindac, oxaprozin, piroxicam, indomethacin, meloxicam, fenoprofen, difunisal, etodolac, ketorolac tromethamine, meclofenamate, nabumetone, salsalate, or any combination of the foregoing.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Synthesis of the Compounds of the Disclosure

The compounds of the disclosure can be synthesized by any method known in the art. For example, the compounds of the disclosure (compounds of Formula (I)) can be synthesized according to Schemes 1, 2, 3, and 4.

Compounds in which A is an optionally substituted fused bicyclic heteroaryl group having 8-10 total ring atoms and 1, 2, or 3 heteroatoms selected from N, O, or S can be synthesized via, e.g., a Suzuki coupling, as shown in Scheme 1, below.

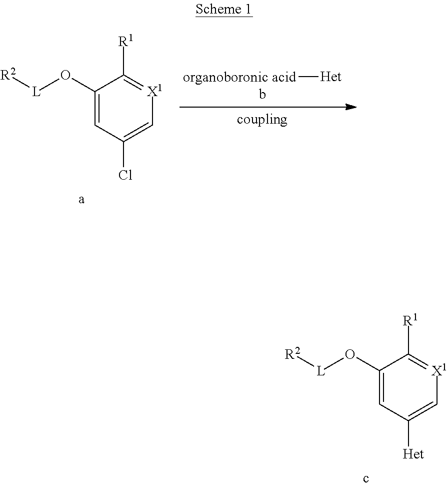

Coupling of a desired phenyl or pyridiyl moiety a with a desired fused bicyclic heteroaryl group (Het) that is conjugated to an organoboronic acid b results in a compound of the disclosure c. Note that while the above scheme shows the “-L-R²” moiety already in place, alternative synthesis can include organoboronic acid aromatic coupling and then introduction of the “O-L-R²” sidechain.

Compounds of the disclosure in which A is

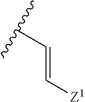

can be synthesized via, e.g., a Horner-Wadsworth-Emmons reaction, as shown in Scheme 2, below.

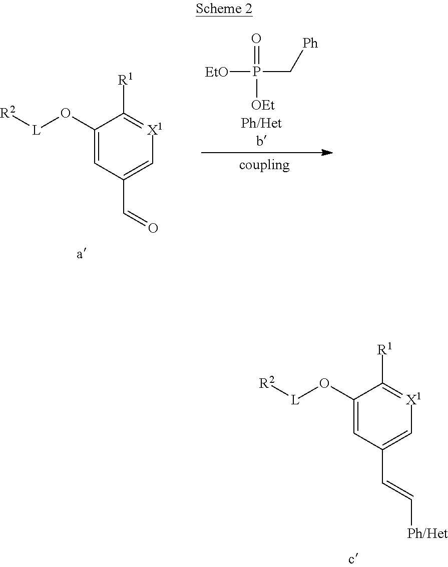

Reaction of a desired aldehyde a′ with a desired phenyl or heteroaryl group (“Ph/Het”) b′ and diethyl benzoylphosphonate results in a compound of the disclosure c′. Note that while the above scheme shows the “O-L-R²” moiety already in place, alternative synthesis can include acid Horner-Wadsworth-Emmons coupling and then introduction of the “O-L-R²” sidechain.

Compounds of the disclosure in which Y¹ is O, S, S═O, or SO₂ can be synthesized, as shown in Scheme 3, below.

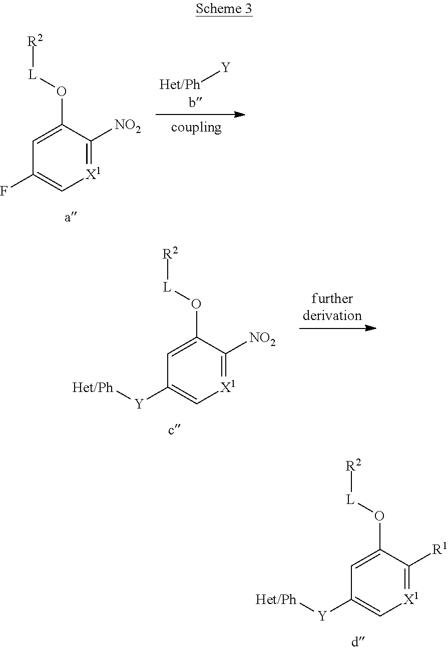

Coupling of a 5-fluoro-2-nitrobenzene moiety a″ (shown with the O-L-R² moiety already attached which can alternatively be inserted later downstream in the synthesis) with a desired heteroaryl or phenyl group (“Het/Ph”) conjugated to a OH or SNa (“Y”) b″ in a nucleophilic aromatic substitution reaction results in compound c″, which can be derivatized at the NO₂ group, the O-L-R² group(s), the X¹ group, and/or the Y group using standard methods known in the art to arrive at a compound of the disclosure, structure d″.

Compounds of the disclosure feature one or two O-L-R² groups which can be synthesized according to Scheme 4, below. Compounds of the disclosure in which X¹ is C—O-L-R² can also be synthesized according to Scheme 4, below.

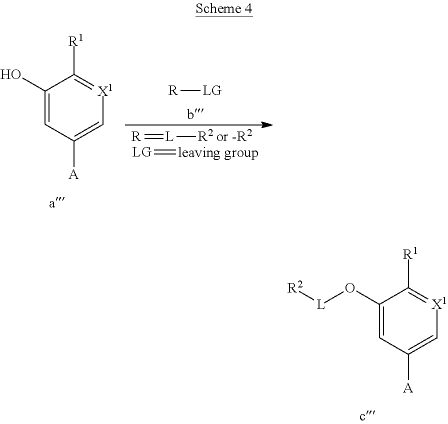

Compounds of Formula (I) can be synthesized using the procedure shown in Scheme 4. Reaction of a hydroxyl-substituted benzene or pyridine compound a′″ with a nucleophilic R derivative compound b′″ produces compounds as described herein, i.e., compounds of Formula (I) c′″.

As used herein, LG refers to any suitable moiety known in the art to be displaceable by a nucleophile. Examples of leaving groups include, but are not limited to, halides (e.g., chloride, bromide, iodide), sulfonates (e.g., tosylate, mesylate, triflate), sulfides (e.g., SCH₃), a carboxylic acid coupling reagent derivative (e.g., N-hydroxsuccinimide, N-hydroxybenzotriazole), an activated carboxylic acid (e.g., acyl chloride), or an acidic hydroxyl group activated via Mitsunobu condition. Nucleophiles are known in the art and include amines, alcohols, and thiols.

The coupling of compounds a′″ and b′″ can be catalyzed or facilitated by appropriate reagents selected based on the precise nature of compounds a and b. For example, when compound b′″ is a phosphoroyl compound (i.e., when R² is a phosphate group), the coupling of compound a′″ and a phosphoroyl compound b′″ can be catalyzed by a base e.g., triethylamine. Occasionally, the coupling reaction may not require a catalyst, or the solvent for the reaction can be the catalyst, e.g., when compound b′″ is a sulfur trioxide pyridine complex and the solvent is pyridine (i.e., when R² is a sulfate group).

### EMBODIMENTS OF THE DISCLOSURE

1. A compound having a structure of Formula (I):

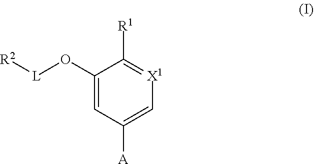

wherein:
A is —Y¹—Z¹ or a fused bicyclic heteroaryl group having 8-10 total ring atoms and 1, 2, or 3 heteroatoms selected from N, O, or S, and the heteroaryl is optionally substituted with 1, 2, or 3 R^{a};
Y¹ is

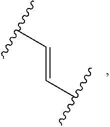 [SMILES: */C=C/*]

O, S, SO, or SO₂;
Z¹ is phenyl or a 5- to 6-membered heteroaryl having 1, 2, or 3 heteroatoms selected from N, O, and S, and the phenyl or heteroaryl is optionally substituted with 1, 2, or 3 R^{a};
X¹ is N, CR³ or C—O-L-R²;
R¹ is C₁₋₅alkyl, C₁₋₅haloalkyl, C₁₋₅hydroxyalkyl, OC₁₋₅alkyl, C₃₋₆cycloalkyl, N(R^{N})₂, C(═O)C₁₋₄alkyl, or C(═CH)C₁₋₄alkyl;
each R² independently is SO₂—R⁴, PO(R⁴)₂, CO₂H, N(R^{N})₂, N(H)—C₂₋₆alkylene-N(R^{N})₂, N(H)—C₂₋₆polyoxyalkylene-N(R^{N})₂, NHCH(R⁵)CO₂H, NH—C₁₋₆alkylene-CH(NH₂)CO₂H, a saccharide, or NH-phenyl wherein the phenyl is optionally substituted with 1, 2, or 3 R^{b};
R³ is halo, C₁₋₆alkoxy, OH, or N(R^{N})₂;
each L independently is C(O), C(O)—Wₙ—C(O), C(O)—Wₙ, or C(O)NH—Wₙ, or when R² is SO₂—R⁴, PO(R⁴)₂, or a saccharide, L can be a bond;
each W independently is CH₂, C₂-alkylene-O, or O—C₂-alkylene;
n is 1, 2, 3, 4, 5, or 6;
each R⁴ independently is OH or NH₂;
each R⁵ independently is H, C₁₋₆alkyl, or C₁₋₆alkylene-R⁶;
each R⁵ independently is CO₂H, CO₂C₁₋₃alkyl, CON(R^{N})₂, OH, OCH₃, N(R^{N})₂, NHC(NH)NH₂, heteroaryl comprising 5 or 6 total ring atoms and 1, 2, or 3 heteroatoms selected from N, O, and S, or phenyl, wherein the heteroaryl or phenyl is optionally substituted with 1, 2, 3, 4, or 5 C₀₋₂alkylene-R^{b} substituents;
each R^{a} independently is halo, C₁₋₆alkyl, CO₂H, CO₂C₁₋₃alkyl, CON(R^{N})₂, OH, OC₁₋₃alkyl, or N(R^{N})₂;
each R^{b} independently is CO₂H, CO₂C₁₋₃alkyl, or OH; and
each R^{N} independently is H or C₁₋₃alkyl;
or a pharmaceutically acceptable salt thereof, provided that when Y¹ is

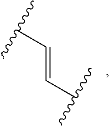 [SMILES: */C=C/*]

Z¹ is unsubstituted phenyl, and R¹ is isopropyl, then X¹ is N or CR³, and R³ is halo, C₁₋₆alkoxy, or N(R^{N})₂.
2. The compound or salt of embodiment 1, wherein A is Y¹—Z¹.
3. The compound or salt of embodiment 2, wherein Y¹ is

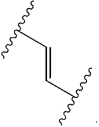 [SMILES: */C=C/*]

4. The compound or salt of embodiment 2, wherein Y¹ is S, SO, or SO₂.
5. The compound or salt of any of embodiments 2 to 4, wherein Z¹ is phenyl.
6. The compound or salt of any one of embodiments 2 to 4, wherein Z¹ is a 5- to 6-membered heteroaryl.
7. The compound or salt of embodiment 6, wherein Z¹ is pyridine, pyrazole, imidazole, or pyrimidine.
8. The compound or salt of any one of embodiments 5 to 7, wherein Z¹ is unsubstituted.
9. The compound or salt of embodiments 5 to 7, wherein Z¹ is substituted with 1 or 2 R^{a} groups.
10. The compound or salt of embodiment 9, wherein each R^{a} is independently halo, C₁₋₃ alkyl, or NH₂.
11. The compound or salt of embodiment 1, wherein A is a fused bicyclic heteroaryl.
12. The compound or salt of embodiment 11, wherein A is a 10-membered heteroaryl.
13. The compound or salt of embodiment 11 or 12, wherein A is isoquinoline, quinoline, or quinazoline.
14. The compound or salt of embodiment 13, wherein A is

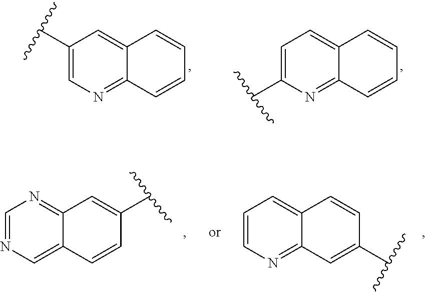 [SMILES: *c1cnc2ccccc2c1 ; *c1ccc2ccccc2n1 ; *c1ccc2cncnc2c1 ; *c1ccc2cccnc2c1]

or
when R² is SO₂—R⁴ or PO(R⁴)₂, A can be

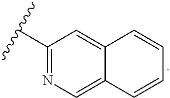 [SMILES: *c1cc2ccccc2cn1]

15. The compound or salt of any one of embodiments 11 to 14, wherein A is unsubstituted.
16. The compound or salt of any one of embodiments 11 to 14, wherein A is substituted with 1 or 2 R^{a} groups.
17. The compound or salt of embodiments 16, wherein each R^{a} is independently halo, C₁₋₃ alkyl, or NH₂.
18. The compound or salt of any one of embodiments 1 to 17, wherein X¹ is N.
19. The compound or salt of any one of embodiments 1 to 17, wherein X¹ is CR³.
20. The compound or salt of embodiments 19, wherein R³ is F or OH.
21. The compound or salt of any one of embodiments 1 to 17, wherein X¹ is C—O-L-R².
22. The compound or salt of embodiment 21, wherein X¹ is C—O—R².
23. The compound or salt of embodiment 22, wherein X¹ is C—OSO₂—R⁴ or C—OPO(R⁴)₂.
24. The compound of or salt of any one of embodiments 1 to 23, wherein R¹ is C₁₋₅alkyl, C₁₋₅haloalkyl, or C₁₋₅hydroxyalkyl.
25. The compound or salt of embodiment 24, wherein R¹ is C₁₋₅alkyl.
26. The compound or salt of embodiment 25, wherein R¹ is C₃alkyl.
27. The compound or salt of any one of embodiments 1 to 23, wherein R¹ is OC₁₋₅alkyl, N(R^{N})₂, or C(═O)C₁₋₄alkyl.
28. The compound or salt of any one of embodiments 1 to 27, wherein L is a bond.
29. The compound or salt of any one of embodiments 1 to 27, wherein L is C(O), C(O)—Wₙ—C(O), C(O)—Wₙ, or C(O)NH—Wₙ.
30. The compound or salt of embodiment 29, wherein each W is independently CH₂ or C₂₋₃ alkylene-O.
31. The compound or salt of embodiment 29 or 30, wherein n is 1 or 2.
32. The compound of or salt of any one of embodiments 1 to 31, wherein R² is SO₂—R⁴ or PO(R⁴)₂.
33. The compound or salt of embodiment 32, wherein R² is SO₂H or PO(OH)₂.
34. The compound or salt of any one of embodiments 1 to 31, wherein R² is N(H)—C₂₋₆ alkylene-N(R^{N})₂, N(H)—C₂₋₆polyoxyalkylene-N(R^{N})₂, NHCH(R⁵)CO₂H, or NH—C₁₋₆alkylene-CH(NH₂)CO₂H.
35. The compound or salt of embodiment 34, wherein R² is N(H)—C₂₋₆alkylene-N(R^{N})₂ or NH—C₁₋₅alkylene-CH(NH₂)CO₂H.
36. The compound or salt of embodiment 35, wherein R² is NHCH(R⁵)CO₂H.
37. The compound or salt of embodiment 36, wherein R⁵ is C₁₋₅alkyl or C₁₋₅alkylene-R⁶.
38. The compound or salt of embodiment 37, wherein R⁶ is CO₂H, CO₂CH₃, CONH₂, OH, OCH₃, NH₂, NHC(NH)NH₂, imidazolyl, phenyl, or 4-hydroxy-phenyl.
39. The compound or salt of any one of embodiments 1 to 31, wherein at least one R² is NH-phenyl.
40. The compound or salt of embodiment 39, wherein the phenyl is substituted with 1 or 2 R^{b} groups.
41. The compound or salt of any one of embodiments 1 to 40, wherein each R^{N} is independently H or CH₃.
42. The compound or salt of embodiment 1 selected from the group consisting of:
(E)-2-isopropyl-5-styrylpyridin-3-yl hydrogen sulfate,
(E)-2-isopropyl-5-styrylpyridin-3-yl dihydrogen phosphate,
3-hydroxy-2-isopropyl-5-(isoquinolin-3-yl)phenyl hydrogen sulfate,
3-hydroxy-2-isopropyl-5-(isoquinolin-3-yl)phenyl dihydrogen phosphate,
3-hydroxy-2-isopropyl-5-(quinolin-3-yl)phenyl hydrogen sulfate,
3-hydroxy-2-isopropyl-5-(quinolin-3-yl)phenyldihydrogen phosphate,
2-isopropyl-5-(quinolin-3-yl)-1,3-phenylenebis(hydrogen sulfate),
2-isopropyl-5-(quinolin-3-yl)-1,3-phenylenebis(dihydrogen phosphate),
(E)-5-(2-fluorostyryl)-2-isopropylpyridin-3-yl hydrogen sulfate,
(E)-5-(2-fluorostyryl)-2-isopropylpyridin-3-yl dihydrogen phosphate,
(E)-5-(3-fluorostyryl)-2-isopropylpyridin-3-yl hydrogen sulfate,
(E)-5-(3-fluorostyryl)-2-isopropylpyridin-3-yl dihydrogen phosphate,
(E)-5-(4-fluorostyryl)-2-isopropylpyridin-3-yl hydrogen sulfate,
(E)-5-(4-fluorostyryl)-2-isopropylpyridin-3-yl dihydrogen phosphate,
5-(2-amino-5-fluoroquinazolin-7-yl)-3-hydroxy-2-isopropylphenyl hydrogen sulfate,
5-(2-amino-5-fluoroquinazolin-7-yl)-3-hydroxy-2-isopropylphenyl dihydrogen phosphate,
5-(2-amino-5-fluoroquinazolin-7-yl)-2-isopropyl-1,3-phenylene bis(hydrogen sulfate);
5-(2-amino-5-fluoroquinazolin-7-yl)-2-isopropyl-1,3-phenylene bis(dihydrogen phosphate);
5-(6-fluoroquinolin-2-yl)-3-hydroxy-2-isopropylphenyl hydrogen sulfate,
5-(6-fluoroquinolin-2-yl)-3-hydroxy-2-isopropylphenyl dihydrogen phosphate,
5-(6-fluoroquinolin-2-yl)-2-isopropyl-1,3-phenylene bis(hydrogen sulfate),
5-(6-fluoroquinolin-2-yl)-2-isopropyl-1,3-phenylene bis(dihydrogen phosphate),
(E)-3-fluoro-2-propyl-5-styrylphenyl hydrogen sulfate,
(E)-3-fluoro-2-propyl-5-styrylphenyl dihydrogen phosphate,
(E)-3-fluoro-2-isopropyl-5-styrylphenyl hydrogen sulfate,
(E)-3-fluoro-2-isopropyl-5-styrylphenyl dihydrogen phsophate,
3-fluoro-2-isopropyl-5-(4-methylquinolin-7-yl)phenyl hydrogen sulfate, and
3-fluoro-2-isopropyl-5-(4-methylquinolin-7-yl)phenyl dihydrogen phosphate.
   43. The compound or salt of embodiment 42 selected from the group consisting of: 2-isopropyl-5-(quinolin-3-yl)-1,3-phenylene bis(dihydrogen phosphate), 2-isopropyl-5-(quinolin-3-yl)-1,3-phenylene bis(hydrogen sulfate), (E)-5-(2-fluorostyryl)-2-isopropylpyridin-3-yl hydrogen sulfate, (E)-5-(2-fluorostyryl)-2-isopropylpyridin-3-yl dihydrogen phosphate, (E)-3-fluoro-2-propyl-5-styrylphenyl hydrogen sulfate, (E)-3-fluoro-2-propyl-5-styrylphenyl dihydrogen phosphate, (E)-3-fluoro-2-isopropyl-5-styrylphenyl hydrogen sulfate, (E)-3-fluoro-2-isopropyl-5-styrylphenyl dihydrogen phosphate, 3-fluoro-2-isopropyl-5-(4-methylquinolin-7-yl)phenyl hydrogen sulfate, and 3-fluoro-2-isopropyl-5-(4-methylquinolin-7-yl)phenyl dihydrogen phosphate.
   44. A pharmaceutical formulation comprising the compound or salt of any one of embodiments 1 to 43 and a pharmaceutically acceptable excipient.
   45. A method of modulating the aryl hydrocarbon receptor (AHR) activity in a subject, comprising contacting the AHR with the compound or salt of any one of embodiments 1 to 43 in an amount effective to modulate the AHR activity.
   46. A method of treating or preventing a disease or disorder associated with aberrant aryl hydrocarbon receptor (AHR) activity in a subject, comprising administering to the subject a therapeutically effective amount of the compound or salt of any one of embodiments 1 to 43.
   47. The method of embodiment 46, wherein the administration is topical.
   48. The method of embodiment 46, wherein the administration is oral or via injection or infusion.
   49. The method of embodiment 46, 47, or 48, wherein the disease or disorder is an inflammatory disease or disorder.
   50. The method of embodiment 46, 47, or 48, wherein the disease or disorder is a disease or disorder of the gastrointestinal tract, skin, lung, central nervous system, pancreas, eye, bones, bone joints, a neuroinflammatory disease, or a neurodegenerative disease.
   51. The method of embodiment 50, wherein the disease or disorder of the gastrointestinal tract is selected from the group consisting of colitis, inflammatory bowel disease, Crohn's disease, celiac disease, necrotizing enterocolitis, irritable bowel syndrome, chronic idiopathic constipation, traveler's diarrhea, and colorectal cancer.
   52. The method of embodiment 50, wherein the disease or disorder of the skin is selected from the group consisting of atopic dermatitis, acne, psoriasis, and vitiligo.
   53. The method of embodiment 50, wherein the disease or disorder of the eye is abnormal eye movements, inflammatory ocular disease, autoimmune ocular disease, hereditary ocular disease, degenerative ocular disease, vascularization ocular disease, dry and wet age-related macular degeneration (“AMD”), Uveitis, retinitis pigmentosa (“RP”), primary open-angle glaucoma (“POAG”), primary congenital glaucoma, Behcet's disease, or Leber congenital amaurosis (“LCA”).
   54. The method of embodiment 50, wherein the disease or disorder of the lung is lung fibrosis, asthma or chronic obstructive pulmonary disease.
   55. The method of embodiment 50, wherein the disease or disorder of the bone joints is osteoporosis, rheumatoid arthritis, or bone cancer.
   56. The method of embodiment 46, 47, or 48, wherein the disease or disorder is diabetes, cancer, a viral infection, or a bacterial infection.
   57. The method of embodiment 56, wherein the viral infection is flavivirus infection or coronavirus infection.
   58. The method of embodiment 56, wherein the bacterial infection is a pulmonary infection, gastrointestinal infection, skin infection, ear infection or a septicemia.
   59. The method of any one of embodiments 46 to 58, further comprising administration of a therapeutic agent.
   60. The method of embodiment 59, wherein the therapeutic agent is an anti-inflammatory agent.
   61. The method of embodiment 60, wherein the anti-inflammatory agent is selected from mesalazine, naproxen, ibuprofen, diclofenac, celecoxib, sulindac, oxaprozin, piroxicam, indomethacin, meloxicam, fenoprofen, difunisal, etodolac, ketorolac tromethamine, meclofenamate, nabumetone, salsalate, or any combination of the foregoing.

### EXAMPLES

The following examples are provided for illustration and are not intended to limit the scope of the invention.

### Preparation of {2-isopropyl-5-[(E)-2-phenylethenyl]pyridin-3-yl}oxidanesulfonic acid (Compound 1)

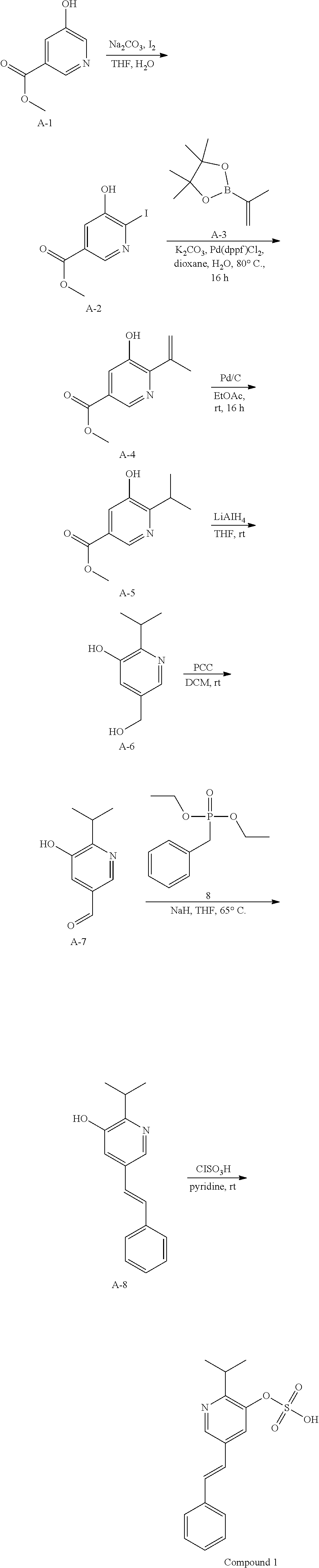

To a solution of methyl 5-hydroxypyridine-3-carboxylate (5.0 g, 0.033 mol) in THF/H₂O (50 ml/50 ml) was added Na₂CO₃ (10.4 g, 0.098 mol) and I₂ (20.8 g, 0.082 mol). The reaction mixture was stirred at 25° C. for 12 h. The solvent was removed by vacuum to crude product which was purified by flash chromatography (eluting with DCM/MeOH from 100/0 to 90/10 in 30 mins) to afford compound A-2. LCMS (ESI): calcd for C₇H₆₁NO₃ [M+H]⁺ m/z 280.0, found 279.8.

To a solution of compound A-2 (705.0 mg, 2.53 mmol) in dioxane/H₂O=10:1 (15 mL) was added A-3 (509.5 mg, 3.03 mmol) and Pd(PPh₃)₄ (292.0 mg, 2.53 mmol) and K₂CO₃ (697.3 mg, 5.05 mmol). The reaction mixture was stirred at 80° C. for 12 h under nitrogen. The solvent was removed by vacuum to crude product which was purified by flash chromatography (eluting with DCM/MeOH from 100/0 to 90/10 in 30 mins) to afford compound A-4. LCMS (ESI): calcd for C₁₀H₁₁NO₃ [M+H]⁺ m/z 194.0, found 194.0.

To a solution of A-4 (134.0 mg, 0.69 mmol) in ethyl acetate (10 mL) was added Pd/C (73.8 mg, 10%). The reaction mixture was stirred at 25° C. for 12 h under 1 atm of hydrogen. The residue was filtered over celite and the filtrate was concentrated under reduced pressure to afford compound A-5, which was used directly for the next step. LCMS (ESI): calcd for C₁₀H₁₃NO₃ [M+H]⁺ m/z 196.0, found 196.0.

To a solution of A-5 (104.0 mg, 0.53 mmol) in THF (5 mL) was added LiAlH₄ (30.3 mg, 0.80 mmol) slowly at 0° C. The reaction mixture was stirred at 25° C. for 2 h. The residue was quenched by water and removed by vacuum to crude product which was purified by flash chromatography (eluting with DCM/MeOH from 100/0 to 80/20 in 20 mins) to afford compound A-6. LCMS (ESI): calcd for C₉H₁₃NO₂ [M+H]⁺ m/z 168.0, found 168.0.

To the solution of A-6 (80.0 mg, 0.48 mmol) in DCM (5 mL) was added PCC (154.7 mg, 0.72 mmol) slowly at 0° C. The reaction mixture was stirred at 25° C. for 4 h. The solvent was removed by vacuum to crude product which was purified by flash chromatography (DCM/MeOH=100/0 to 80/20) to afford compound A-7. LCMS (ESI): calcd for C₉H₁₁NO₂[M+H]⁺ m/z 166.0, found 166.0.

To a solution of diethyl benzylphosphonate (134.7 mg, 0.59 mmol) in THF (3 mL) was added NaH (19.0 mg, 0.79 mmol, 60%) at 0° C. After 2 h, the A-7 (65.0 mg, 0.39 mmol) in THF was added dropwise at 0° C. The reaction mixture stirred at 25° C. for 16 h. After completion, the mixture was poured onto ice, extracted with EtOAc (10 mL×3), the combined organic layer was then washed with sat. NaCl (20 mL) and dried over anhydrous Na₂SO₄. After filtration, the solvent was evaporated under reduced pressure and the crude product was purified by silica column chromatography (PE/EtOAc=100/0 to 50/50) to afford compound A-8.

A solution of A-8 (100.0 mg, 0.42 mmol) in pyridine (5 ml) was added ClSO₃H (193.9 mg, 1.68 mmol) dropwise. The reaction mixture was stirred at 25° C. for 2 hours. The desired mass was detected on LC-MS. The solvent was removed under reduce pressure. The residue was purified by C₁₈ column (eluting with ACN-H₂O (0.1% HCOOH) from 0:100 to 50:50 in 30.0 min) to afford compound 1. LCMS (ESI): calcd for C₁₆H₁₇NO₄S [M−H]⁻ m/z 318.0, found 317.9. ¹H NMR (400 MHz, DMSO-d₆, ppm) 8.55 (s, 1H), 8.23 (s, 1H), 7.66 (d, J=7.2 Hz, 2H), 7.41 (t, J=7.6 Hz, 2H), 7.36-7.30 (m, 3H), 3.61-3.54 (m, 1H), 1.24 (d, J=7.2 Hz, 6H).

### Preparation of Preparation of [3-hydroxy-2-isopropyl-5-(isoquinolin-3-yl)phenyl]oxidanesulfonic acid (Compound 2)

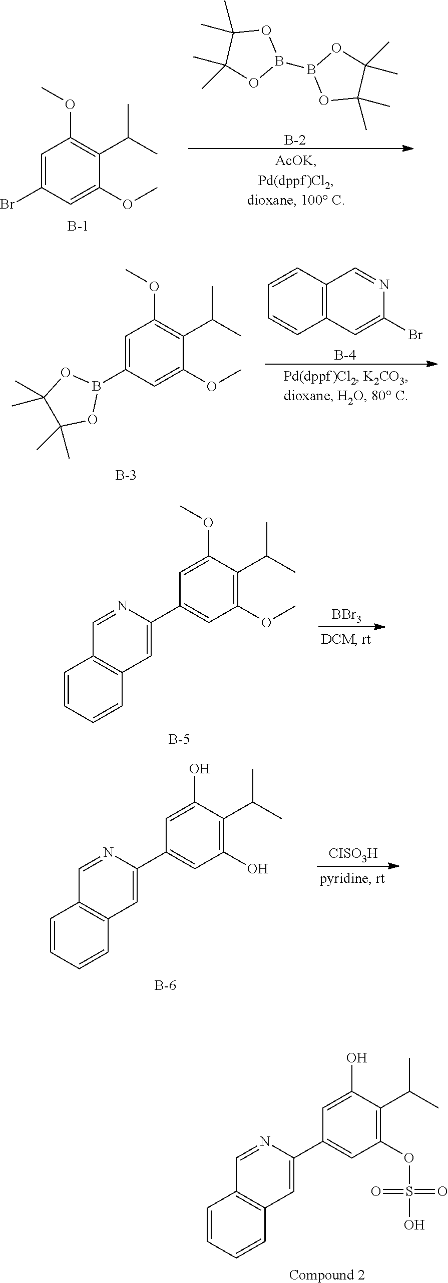

To a solution of 5-bromo-2-isopropyl-1,3-dimethoxybenzene (200.0 mg, 0.77 mmol) in 1,4-dioxane (5 ml) was added B-2 (235.2 mg, 0.927 mmol), Pd(dppf)Cl₂ (56.5 mg, 0.077 mmol) and potassium acetate (151.5 mg, 1.54 mmol). The reaction mixture was stirred at 100° C. for 16 h under nitrogen. The solvent was removed by vacuum. The residue was purified by silica gel chromatography (eluting with PE/EtOAc from 50/1 to 10/1 in 30 mins) to afford B-3. ¹H NMR (400 MHz, DMSO-d₆, ppm) 6.84 (s, 2H), 3.79-3.71 (m, 6H), 3.62-3.46 (m, 1H), 1.29 (s, 12H), 1.20 (d, J=7.2 Hz, 6H).

To a solution of 2-(4-isopropyl-3,5-dimethoxyphenyl)-4,4,5,5-tetramethyl-1,3,2-dioxaborolane (120.0 mg 0.39 mmol) in 1,4-dioxane/H₂O=3:1 (8 ml) was added 3-bromoisoquinoline (97.8 mg, 0.47 mmol), K₂CO₃ (109.9 mg, 0.78 mmol) and Pd(dppf)Cl₂ (28.7 mg, 0.039 mmol). The reaction mixture was stirred at 80° C. for 12 h under nitrogen. The solvent was removed by vacuum. The residue was purified by silica gel chromatography (eluting with PE/EtOAc from 50:1 to 10:1 in 30 mins) to afford B-5. LCMS (ESI): calcd for C₂₀H₂₁NO₂ [M+H]⁺ m/z=308.1, found 307.9. ¹H NMR (400 MHz, DMSO-d₆, ppm): 9.40 (s, 1H), 8.47 (s, 1H), 8.03 (d, J=8.2 Hz, 1H), 7.86-7.62 (m, 3H), 7.49 (s, 2H), 3.90 (s, 6H), 3.65-3.51 (m, 1H), 1.27 (d, J=7.2 Hz, 6H).

To a solution of B-5 (100.0 mg, 0.325 mmol) in DCM (5 ml) was added BBr₃ (0.5 ml, 1M in DCM) dropwise at 0° C. The reaction mixture was stirred at 25° C. for 6 h. The solvent was removed under reduce pressure. The solution was added aq. NaHCO₃ (10 mL) and extracted with EtOAc (10 mL×3). The organic phases were combined and washed with brine, dried over Na₂SO₄, filtered and concentrated in vacuo. The solvent was removed under reduce pressure. The residue was purified by prep-HPLC (columns: Gemini 5 um C18 150×21.2 mm, mobile phase: ACN-H₂O (0.05% NH₃·H₂O), gradient: 35-50, 9.0 min) to afford B-6. LCMS (ESI): calcd for C₁₈H₁₇NO₂ [M+H]⁺ m/z 280.1, found 280.1. ¹H NMR (400 MHz, DMSO-d6, ppm): 9.34 (s, 1H), 9.18 (s, 2H), 8.16-8.06 (m, 1H), 8.03 (d, J=8.2 Hz, 2H), 7.77 (t, J=7.2 Hz, 1H), 7.64 (t, J=7.6 Hz, 1H), 7.13 (s, 2H), 3.55-3.42 (m, 1H), 1.28 (d, J=7.1 Hz, 6H).

To a solution of 2-isopropyl-5-(isoquinolin-3-yl)benzene-1,3-diol (100.0 mg, 0.358 mmol) in pyridine (5 ml) was added ClSO₃H (207.6 mg, 1.79 mmol). The reaction mixture was stirred at 25° C. for 2 h. The desired mass was detected on LC-MS. The solvent was removed under reduce pressure. The residue was purified by C₁3 column (eluting with ACN-H₂O (0.1% NH₃·H₂O from 0:100 to 50:50 in 30.0 min) to afford compound 2. LCMS (ESI): calcd for C₁₈H₁₇NO₅S [M+H]⁻ m/z 360.1, found 360.1. ¹H NMR (400 MHz, DMSO-d₆, ppm) 9.51 (s, 1H), 9.46 (s, 1H), 8.25 (d, J=7.6 Hz, 2H), 8.16 (d, J=8.4 Hz, 1H), 7.90 (t, J=7.6 Hz, 1H), 7.75 (t, J=7.6 Hz, 1H), 7.61 (d, J=1.6 Hz, 1H), 7.33 (s, 1H), 3.58-3.55 (m, 1H), 1.28 (d, J=7.2 Hz, 6H).

### Preparation [3-hydroxy-2-isopropyl-5-(quinolin-3-yl)phenyl]oxidanesulfonic acid (Compound 3)

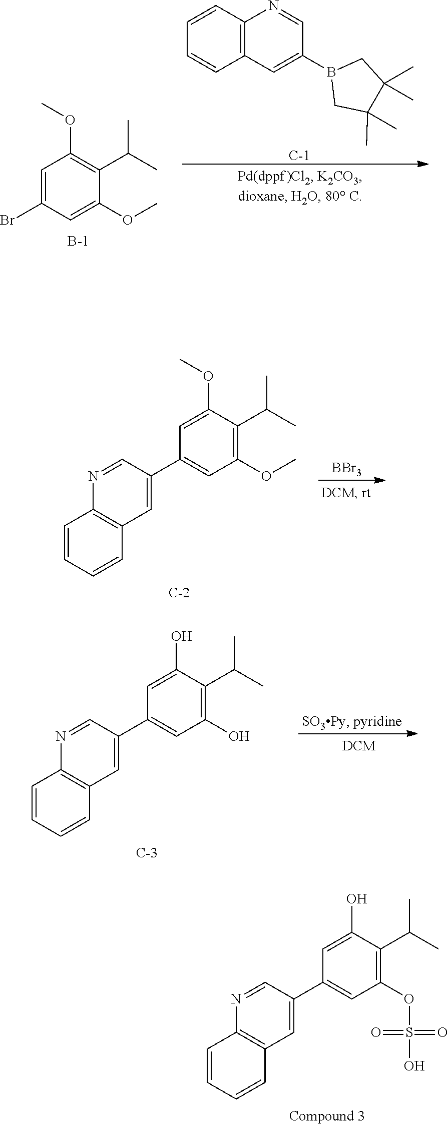

To a solution of 5-bromo-2-isopropyl-1,3-dimethoxybenzene (180.0 mg, 0.69 mmol) in 1,4-dioxane/H₂O (4 mL/4 mL) was added 2-(4,4,5,5-tetramethyl-1,3,2-dioxaborolan-2-yl)quinoline (212.7 mg, 0.83 mmol), Pd(dppf)Cl₂ (50.8 mg, 0.069 mmol) and K₂CO₃ (191.7 mg, 1.39 mmol). The reaction mixture was stirred at 100° C. for 16 h under nitrogen. The LCMS showed the desired MS was detected. The solvent was removed by vacuum. The residue was extracted with EtOAc (200 mL), the organic layer was washed with brine, dried over Na₂SO₄, filtered and concentrated to get crude product which was purified by flash chromatography (eluting with PE/EtOAc from 100/0 to 40/60 in 30 mins) to afford C-2. LCMS (ESI): calcd for C₂₀H₂₁NO₂ [M+H]⁺ m/z 308.1, found 308.2.

To a solution of C-2 (160.0 mg, 0.56 mmol) in DCM (2 ml) was added tribromoborane (0.6 ml, 1 M in DCM) dropwise at −78° C. The reaction mixture was stirred at 25° C. for 12 h. The LCMS showed the desired MS was detected. The solution was added aq. NaHCO₃ and extracted with EtOAc (10 mL×3). The organic phases were combined and washed with brine, dried over Na₂SO₄, filtered and concentrated in vacuo. The residue was purified by prep-HPLC (columns: Gemini 5 um C18 150×21.2 mm, mobile phase: ACN-H₂O (0.1% FA), gradient: 35-95.12 min) to afford C-3. LCMS (ESI): calcd for C₁₈H₁₇NO₂ [M+H]⁺ m/z 280.1, found 280.2. ¹H NMR (400 MHz, DMSO-d₆, ppm) 9.34 (s, 2H), 9.03 (d, J=2.4 Hz, 1H), 8.37 (d, J=2.0 Hz, 1H), 8.09-7.98 (m, 2H), 7.81-7.71 (m, 1H), 7.68-7.59 (m, 1H), 6.68 (s, 2H), 3.55-3.44 (m, 1H), 1.28 (d, J=8.0 Hz, 6H).

To a solution of C-3 (100.0 mg, 0.358 mmol) in DCM (5 ml) were added SO₃·Py (284.8 mg, 1.79 mmol) and pyridine (452.5 mg, 5.73 mmol). The reaction mixture was stirred at 25° C. for 6 hours. The LCMS showed the desired MS was detected. The desired mass was detected on LC-MS. The solvent was removed under reduce pressure. The residue was purified by C₁a column (eluting with ACN-H₂O (0.1% NH₃·H₂O) from 0:100 to 50:50 in 30.0 min) to afford compound 3. LCMS (ESI): calcd for C₁₈H₁₇NO₅S [M+H]⁻ m/z 360.0, found 360.2. ¹H NMR (400 MHz, CD₃OD, ppm) 9.14 (d, J=2.0 Hz, 1H), 8.65 (d, J=1.6 Hz, 1H), 8.06 (d, J=8.4 Hz, 2H), 7.81 (t, J=7.6 Hz, 1H), 7.68 (t, J=7.6 Hz, 1H), 7.45 (s, 1H), 6.98 (s, 1H), 3.72-3.62 (m, 1H), 1.38 (d, J=6.8 Hz, 6H).

### Preparation of 2-isopropyl-3-(phosphonooxy)-5-(quinolin-3-yl)phenoxyphosphonic acid (Compound 4)

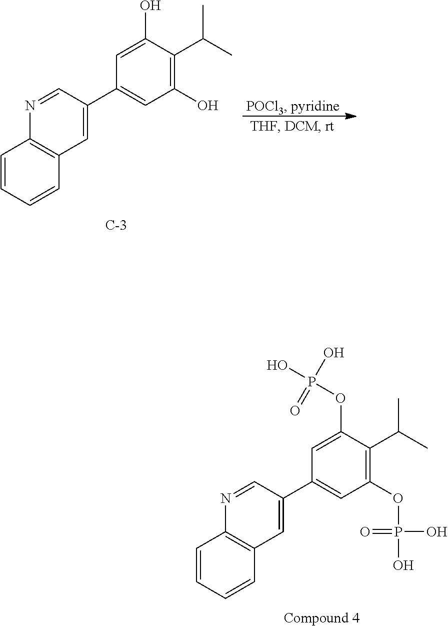

To a solution of 2-isopropyl-5-(quinolin-3-yl)benzene-1,3-diol (400.0 mg, 1.43 mmol) in THF/DCM (1:1, 10 ml) were added POCl₃ (724.6 mg, 4.73 mmol) and pyridine (869.4 mg, 8.59 mmol) dropwise. The reaction mixture was stirred at 25° C. for 2 hours. The desired mass was detected on LC-MS. The solvent was removed under reduce pressure. The residue was purified by C₁₈ column (eluting with ACN-H₂O (0.1% NH₃·H₂O) from 0:100 to 50:50 in 30.0 min) to afford compound 4. LCMS (ESI): calcd for C₈H₁₉NO₈P₂ [M+H]⁻ m/z 440.0, found 440.2. ¹H NMR (400 MHz, DMSO-d₆, ppm) 9.09 (s, 1H), 8.46 (s, 1H), 8.06 (dd, J=15.6, 8.0 Hz, 2H), 7.75 (t, J=7.6 Hz, 1H), 7.62 (t, J=7.2 Hz, 1H), 7.54 (s, 2H), 3.54-3.51 (m, J=7.2 Hz, 1H), 1.27 (d, J=6.4 Hz, 6H).

### Preparation of {5-[(E)-2-(2-fluorophenyl) ethenyl]-2-isopropylpyridin-3-yl}oxidanesulfonic acid (Compound 5)

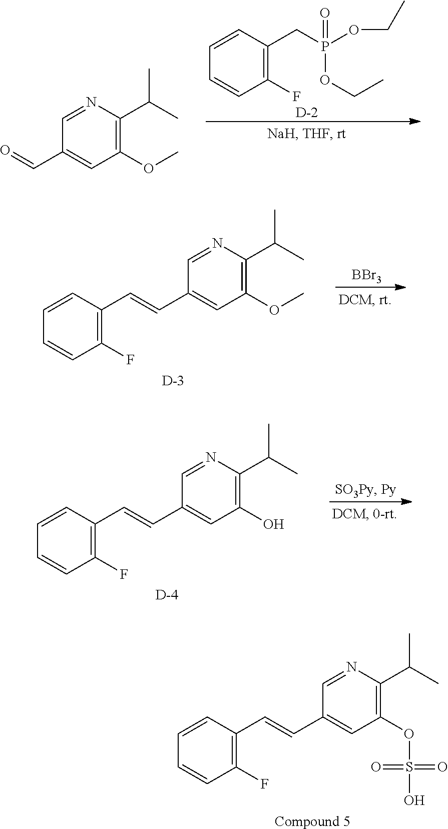

To a solution of diethyl [(2-fluorophenyl)methyl]phosphonate (131.9 mg, 0.536 mmol) in THE (5 mL) was added NaH (26.8 mg, 0.670 mmol, 60% in oil), then the reaction mixture was stirred at 25° C. for 1 h. The mixture was added 6-isopropyl-5-methoxypyridine-3-carbaldehyde (80.0 mg, 0.446 mmol) then the reaction mixture was stirred at 25° C. for 2 h. The mixture was added water (10 mL) and extracted with EtOAc (20 mL×3). The organic phases were combined and washed with brine, dried over Na₂SO₄, filtered out and concentrated in vacuo. The solvent was removed under reduce pressure. The residue was purified by silica gel chromatography (eluting with PE/EtOAc from 100/0 to 95/5 in 20 mins) to afford D-3. LCMS (ESI): calcd for C₁₇H₁₈FNO [M+H]⁺ m/z 272.1, found 272.1.

To a solution of D-3 (60.0 mg, 0.221 mmol) in DCM (5 mL) was added BBr₃ (554.0 mg, 2.21 mmol), then the mixture was stirred at 25° C. for 16 h. The solution was added water (10 mL) and extracted with DCM (10 mL×3). The organic phases were combined and washed with aq. NaHCO₃ and brine, dried over Na₂SO₄, filtered out and concentrated in vacuo. The residue was purified by prep-HPLC (eluting with H₂O (0.1% NH₃·H₂O)/MeCN from 70/30 to 5/95 in 40 mins) to afford D-4. LCMS (ESI): calcd for C₁₆H₁₆FNO [M+H]⁺ m/z 258.1, found 258.0. ¹H NMR (400 MHz, DMSO-d₆, ppm) 9.83 (s, 1H), 8.18 (d, J=1.8 Hz, 1H), 7.85-7.78 (m, 1H), 7.36-7.29 (m, 2H), 7.29-7.12 (m, 4H), 3.41-3.35 (m, 1H), 1.18 (d, J=6.8 Hz, 6H).

To a solution of D-4 (200.0 mg, 0.777 mmol) in DCM (5 mL) were added pyridine (122.8 mg, 1.55 mmol) and SO₃Py (618.0 mg, 7.77 mmol) at 0° C. The reaction mixture was stirred at 25° C. for 2 h. The desired mass was detected on LC-MS. the solvent was removed under reduce pressure. The residue was purified by C₁₈ column (eluting with ACN-H₂O (0.1% NH₃·H₂O) from 20:80 to 60:40 in 30.0 min) to afford compound 5. LCMS (ESI): calced for C₁₆H₁₇FNO₄S [M+H]⁺ ms/z 338.2, found 338.2. ¹H NMR (400 MHz, CD₃OD, ppm) 8.48 (s, 1H), 8.37 (s, 1H), 7.73 (t, J=7.6 Hz, 1H), 7.46 (d, J=16.8 Hz, 1H), 7.36-7.27 (m, 2H), 7.20 (t, J=7.6 Hz, 1H), 7.13 (dd, J=10.8, 8.4 Hz, 1H), 3.72 (hept, J=7.2 Hz, 1H), 1.33 (d, J=7.2 Hz, 6H).

### Preparation of {5-[(E)-2-(4-fluorophenyl) ethenyl]-2-isopropylpyridin-3-yl}oxidanesulfonic acid (Compound 6)

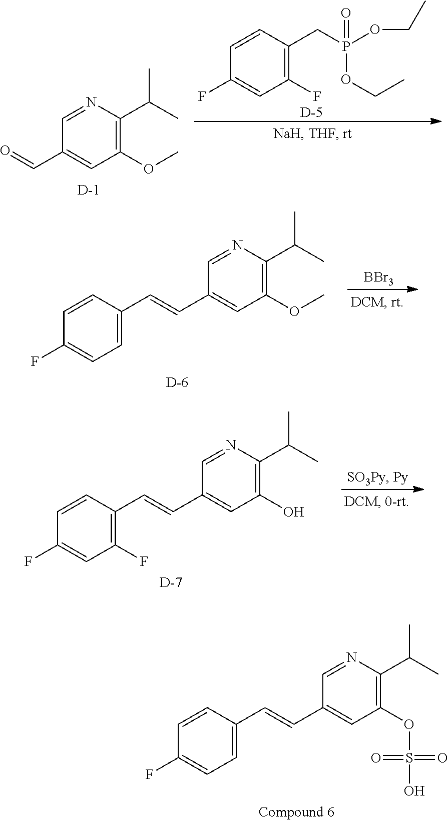

To a solution of diethyl [(4-fluorophenyl)methyl]phosphonate (54.0 mg, 0.218 mmol) in THF (5 mL) was added NaH (60%) (16.0 mg, 0.338 mmol). After 30 min, 6-isopropyl-5-methoxypyridine-3-carbaldehyde (30.0 mg, 0.167 mmol) was added. The reaction mixture was stirred at 25° C. for 2 h. LCMS showed the desired MS was detected. The mixture was added water (10 mL) and extracted with EtOAc (20 mL×3). The organic phases were combined and washed with brine, dried over Na₂SO₄, filtered out and concentrated in vacuo. The residue was purified by silica gel chromatography (eluting with PE/EtOAc from 100/0 to 95/5 in 20 mins) to afford D-6. LCMS (ESI): calcd for C₁₇H₉FNO [M+H]⁺ m/z 272.1, found 272.1.

To a solution of D-6 (40.0 mg, 0.133 mmol) in DCM (5 mL) was added BBr₃ (332.0 mg, 1.33 mmol), then the mixture was stirred at 25° C. for 16 h. The solution was added water (10 mL) and extracted with DCM (10 mL×3). The organic phases were combined and washed with NaHCO₃ (aq.) and brine, dried over Na₂SO₄, filtered out and concentrated in vacuo. The solvent was removed under reduce pressure. The residue was purified by prep-HPLC (columns: Gemini 5 um C18 150×21.2 mm, mobile phase: ACN-H₂O (0.1% FA), gradient: 20-95, 10.0 min) to afford D-7. LCMS (ESI): calcd for C₁₆H₁₇FNO [M+H]⁺ m/z 258.1, found 258.1. ¹H NMR (400 MHz, DMSO-d₆, ppm) 9.78 (s, 1H), 8.16 (d, J=1.8 Hz, 1H), 7.66 (m, 2H), 7.27 (d, J=1.8 Hz, 1H), 7.21 (t, J=8.8 Hz, 2H), 7.14 (s, 2H), 3.36 (m, 1H), 1.17 (d, J=6.8 Hz, 6H).

SO₃Py (804.0 mg, 5.05 mmol) and pyridine (160.0 mg, 2.02 mmol) was added to a solution of E-3 (260.0 mg, 1.01 mmol) in DCM (6 ml). Then the mixture was stirred at rt for 16 h. The desired mass was detected on LC-MS. The solvent was removed under reduced pressure. The residue was purified by prep-HPLC (columns: Gemini 5 um C18 150×21.2 mm, mobile phase: H₂O-ACN (0.05% NH₃·H₂O), gradient: 80-20, 10.0 min) to afford compound 6. LCMS (ESI): calced for C₁₆H₁₇FNO₄S [M+H]⁺ ms/z 338.2, found 338.1. ¹H NMR (400 MHz, DMSO-d₆, ppm) 8.53 (s, 1H), 8.20 (s, 1H), 7.72 (dd, J=8.8, 5.6 Hz, 2H), 7.39-7.30 (m, 2H), 7.29-7.21 (m, 2H), 3.64-3.48 (m, 1H), 1.24 (d, J=6.8 Hz, 6H).

### Preparation of [5-(2-amino-5-fluoroquinazolin-7-yl)-3-hydroxy-2-isopropylphenyl]oxidanesulfonic acid (Compound 7)

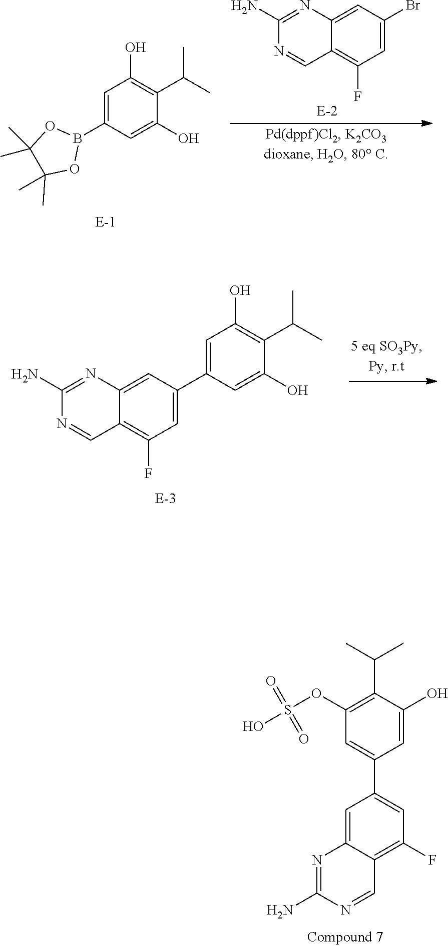 [SMILES: CC(C)c1c(O)cc(B2OC(C)(C)C(C)(C)O2)cc1O ; Nc1ncc2c(F)cc(Br)cc2n1 ; CC(C)c1c(O)cc(-c2cc(F)c3cnc(N)nc3c2)cc1O ; CC(C)c1c(O)cc(-c2cc(F)c3cnc(N)nc3c2)cc1OS(=O)(=O)O]

To a solution of 2-isopropyl-5-(4,4,5,5-tetramethyl-1,3,2-dioxaborolan-2-yl)benzene-1,3-diol (1.0 g, 3.5971 mmol) in 1,4-dioxane (50 mL) and H₂O (10 mL) were added 7-bromo-5-fluoroquinazolin-2-amine (870.9 mg, 3.5971 mmol), Pd(dppf)Cl₂ (262.9 mg, 0.3597 mmol) and K₂CO₃ (994.3 mg, 7.1942 mmol). The reaction mixture was stirred at 80° C. for 12 h under N₂. The desired mass was detected on LC-MS. The mixture reaction was quenched with water, extracted with EtOAc, the combined organic phase was washed with brine, dried over anhydrous Na₂SO₄, filtered and concentrated. The residue was purified by silica gel chromatography (eluting with PE/(EtOAc:DCM=1:1) form 50:50 to 10:90 in 30 mins) to afford E-3. LCMS (ESI): calcd for C₁₇H₁₆FN₃O₂ [M+H]⁺ ms/z 314.1, found 314.2. ¹H NMR (400 MHz, DMSO-d₆, ppm) 9.31 (s, 2H), 9.22 (s, 1H), 7.25 (s, 1H), 7.13 (s, 2H), 7.04 (d, J=11.2 Hz, 1H), 6.59 (s, 2H), 3.47 (m, 1H), 1.26 (d, J=7.2 Hz, 6H).

To a solution of E-3 (150.0 mg, 0.4787 mmol) in pyridine (5 mL) was added SO₃Py (380.95 mg, 2.3935 mmol), then the mixture was stirred at 25° C. for 12 h. The desired mass was detected on LC-MS. The solvent was removed under reduce pressure. The residue was purified by prep-HPLC (columns: Gemini 5 um C18 150×21.2 mm, mobile phase: H₂O-ACN (0.05% NH₃·H₂O), gradient: 85-5, 11.0 min) to afford [5-(2-amino-5-fluoroquinazolin-7-yl)-3-hydroxy-2-isopropylphenyl]oxidanesulfonic acid. LCMS (ESI): calcd for C₁₇H₁₈FN₃O₅S [M−H]⁻ m/z 392.1, found 392.3. ¹H NMR (400 MHz, DMSO-d₆, ppm) 9.46 (s, 1H), 9.27 (s, 1H), 7.31 (s, 2H), 7.26 (d, J=1.6 Hz, 1H), 7.20 (s, 1H), 7.11-7.07 (m, 2H), 6.94 (s, 1H), 6.85 (d, J=2.0 Hz, 1H), 3.55-3.47 (m, 1H), 1.26 (d, J=7.2 Hz, 6H).

### Preparation of [5-(6-fluoroquinolin-2-yl)-3-hydroxy-2-isopropylphenyl]oxidanesulfonic acid (Compound 8)

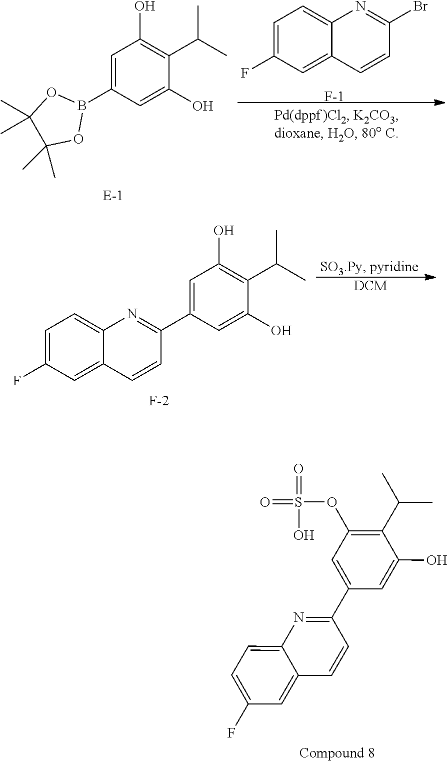

To a solution of 2-isopropyl-5-(4,4,5,5-tetramethyl-1,3,2-dioxaborolan-2-yl)benzene-1,3-diol (541.4 mg, 1.95 mmol) in 1,4-dioxane/H₂O (16 mL/4 mL) were added 2-bromo-6-fluoroquinoline (400.0 mg, 1.77 mmol), Pd(dppf)Cl₂ (129.5 mg, 0.18 mmol) and K₂CO₃ (489.2 mg, 3.54 mmol). The reaction mixture was stirred at 80° C. for 12 h under nitrogen. The desired mass was detected on LC-MS. The solvent was removed by vacuum. The residue was quenched with water, extracted with EtOAc, the combined organic phase was washed with brine, dried over anhydrous Na₂SO₄, filtered and concentrated. The residue was purified by silica gel chromatography (eluting with PE/EtOAc from 100/0 to 40/60 in 30 mins) and C₁₈ column (eluting with 0.5% NH₃·H₂O/MeCN from 100:0 to 20:80 in 30 mins) to afford F-2. LCMS (ESI): calcd for C₁₈H₁₆FNO₂ [M+H]⁺ ms/z 298.1, found 298.3. ¹H NMR (400 MHz, DMSO-d₆, ppm) 9.29 (s, 2H), 8.40 (d, J=8.8 Hz, 1H), 8.04 (dd, J=9.2, 5.6 Hz, 1H), 7.86 (d, J=8.8 Hz, 1H), 7.79 (dd, J=9.2, 2.8 Hz, 1H), 7.69-7.64 (m, 1H), 7.14 (s, 2H), 3.55-3.44 (m, 1H), 1.28 (d, J=7.2 Hz, 6H).

To a solution of F-2 (300.0 mg, 1.074 mmol) in DCM (5 ml) were added SO₃·Py (854.4 mg, 5.37 mmol) and pyridine (1357.5 mg, 17.19 mmol). The reaction mixture was stirred at 25° C. for 6 hours. The desired mass was detected on LC-MS. The solvent was removed under reduce pressure. The residue was purified by C₁₈ column (eluting with ACN-H₂O (0.1% NH₃·H₂O from 0:100 to 60:40 in 30 mins) from 0:100 to 50:50 in 30.0 min) to afford compound 8. LCMS (ESI): calcd for C₁₈H₁₆FNO₅S [M+H]⁻ m/z 378.0, found 378.3. ¹H NMR (400 MHz, DMSO-d₆, ppm) 9.46 (s, 1H), 8.46 (d, J=8.8 Hz, 1H), 8.10 (dd, J=9.2, 5.6 Hz, 1H), 7.91 (d, J=8.8 Hz, 1H), 7.82 (dd, J=9.6, 2.8 Hz, 1H), 7.72-7.65 (m, 2H), 7.44 (d, J=1.6 Hz, 1H), 3.59-3.51 (m, 1H), 1.27 (d, J=6.8 Hz, 6H).

### Preparation of {3-fluoro-5-[(E)-2-phenylethenyl]-2-propylphenyl}oxidanesulfonic acid (Compound 9)

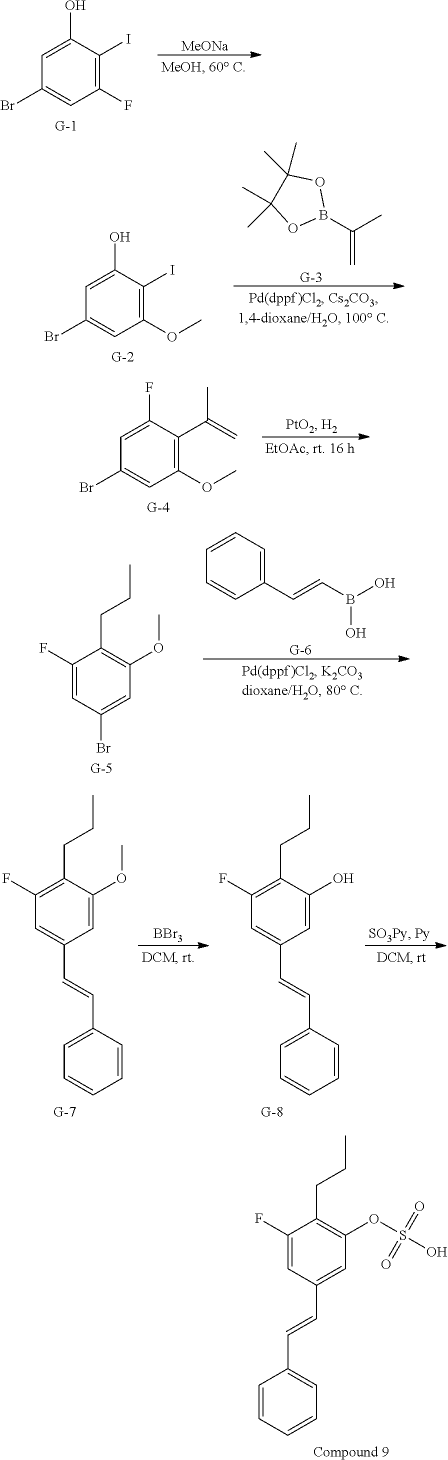

To 5-bromo-1,3-difluoro-2-iodobenzene (2.0 g, 6.3 mmol) in MeOH (20 ml) was added MeONa (30% in MeOH, 2.1 ml). The reaction was stirred at 60° C. for 16 h. The reaction solvent was removed by vacuum to afford white solid. The solvent was diluted with DCM (20 mL) and water (20 mL). The aqueous phase was extracted with DCM (20 mL). The organic phase was combined and washed with brine, dried over anhydrous NaSO₄ and concentrated to get crude product which was purified by flash chromatography (eluting with PE/EtOAc from 100/0 to 98/2 in 30 mins) to afford G-2. ¹H NMR (400 MHz, CDCl₃, ppm) 6.91 (dd, J=7.2. 2.0 Hz, 1H), 6.76 (t, J=1.6 Hz, 1H), 3.90 (s, 3H).

To a solution of G-2 (2.0 g, 6.04 mmol) in 1,4-dioxane/H₂O (40 mL/4 mL) was added G-3 (1.5 g, 9.07 mmol), Pd(dppf)Cl₂ (442.2 mg, 0.604 mmol) and K₂CO₃ (3.9 g, 12.08 mmol). The reaction mixture was stirred at 100° C. for 16 h under nitrogen. The solvent was removed by vacuum. The residue was purified by flash chromatography (eluting with PE/EA from 100/0 to 98/2 in 30 mins) to afford G-4. ¹H NMR (400 MHz, CDCl₃, ppm) 6.90 (dd, J=8.4, 1.6 Hz, 1H), 6.82 (t, J=1.6 Hz 1H), 5.35 (dd, J=3.2, 1.6 Hz, 1H), 4.97-4.96 (m, 1H), 3.82 (s, 3H), 2.01 (s, 3H).

To a solution of G-4 (200.0 mg, 0.816 mmol) in EtOAc (5 ml) was added Pt/C (20.0 mg, 10 wt %). The reaction mixture was stirred at 25° C. for 16 h under H₂ (1 atm). The solvent was removed by vacuum. The residue was purified by flash chromatography (eluting with PE/EA from 100/0 to 95/5 in 30 mins) to afford G-5. ¹H NMR (400 MHz, CDCl₃, ppm) 6.84 (dd, J=8.8, 1.6 Hz, 1H), 6.77 (t, J=1.6 Hz 1H), 3.83-3.80 (m, 3H), 2.55 (td, J=7.6, 1.6 Hz, 2H), 1.51 (dt, J=14.8, 7.6 Hz, 2H), 0.91 (t, J=8.0 Hz, 3H).

To a solution of G-5 (50.0 mg, 0.202 mmol) in 1,4-dioxane/H₂O (2 mL/0.2 mL) was added [(E)-2-phenylethenyl]boranediol (44.9 mg, 0.304 mmol), Pd(dppf)Cl₂ (11.8 mg, 0.020 mmol) and K₂CO₃ (55.9 mg, 0.405 mmol). The reaction mixture was stirred at 80° C. for 16 h under nitrogen. The LCMS showed the desired MS was detected. The solvent was removed by vacuum. The residue was purified by flash chromatography (eluting with PE/EA from 100/0 to 95/5 in 30 mins) to afford G-7. ¹H NMR (400 MHz, CD₃OD, ppm) 7.56 (d, J=8.0 Hz, 2H), 7.37-7.33 (m, 2H), 7.27-7.23 (m, 1H), 7.16-7.08 (m, 2H), 6.95-6.86 (m, 2H), 3.90 (s, 3H), 2.664-2.59 (m, 2H), 1.60-1.51 (m, 2H), 0.94 (t, J=8.0 Hz, 3H).

To 1-fluoro-3-methoxy-5-[(E)-2-phenylethenyl]-2-propylbenzene (45.0 mg, 0.166 mmol) was added BBr₃ (1M in DCM, 5 mL), then the reaction was stirred at 25° C. for 16 h. The solution was added water (10 mL) and extracted with DCM (10 mL×3). The organic phases were combined and washed with aq. NaHCO₃ and brine, dried over Na2SO4, filtered out and concentrated in vacuo. The solvent was removed under reduce pressure to afford crude. The crude was purified on reversed-phase column directly (eluting with ACN-H₂O (0.1% FA) from 0 to 100 in 30 mins) to afford G-8. LCMS (ESI): calcd for C₁₇H₁₇FO [M+H]⁺ m/z 257.2, found 257.2. ¹H NMR (400 MHz, DMSO-d₆, ppm) 9.79 (s, 1H), 7.59 (d, J=7.6 Hz, 2H), 7.37 (t, J=7.6 Hz, 2H), 7.27 (t, J=7.2 Hz, 1H), 7.13-7.09 (m, 2H), 6.91-6.79 (m, 2H), 1.59-1.42 (m, 2H), 1.24 (t, J=6.4 Hz, 2H), 0.89 (t, J=8.0 Hz, 3H).

To a solution of G-8 (200.0 mg, 0.781 mmol) in DCM (5 mL) was added Py (617.2 mg, 7.81 mmol) and SO₃Py (1.24 g, 7.81 mmol), then the reaction mixture was stirred at 25° C. for 2 h. The desired mass was detected on LC-MS. The solvent was removed under reduce pressure. The residue was purified by C₁a column (eluting with ACN-H₂O (0.1% NH₃·H₂O) from 20:80 to 60:40 in 30.0 min) to afford compound 9. LCMS (ESI): calcd for C₁₇H₁₈FO [M+H]⁻ ms/z 334.9, found 334.9. ¹H NMR (400 MHz, CD₃OD, ppm) 7.54 (d, J=6.4 Hz, 3H), 7.33 (t, J=7.6 Hz, 2H), 7.22 (dd, J=16.4, 9.2 Hz, 1H), 7.10 (m, 3H), 2.71 (t, J=7.6 Hz, 2H), 1.68-1.55 (m, 2H), 0.96 (t, J=7.4 Hz, 3H).

### Preparation of {3-fluoro-2-isopropyl-5-[(E)-2-phenylethenyl]phenyl}oxidanesulfonic acid (Compound 10)

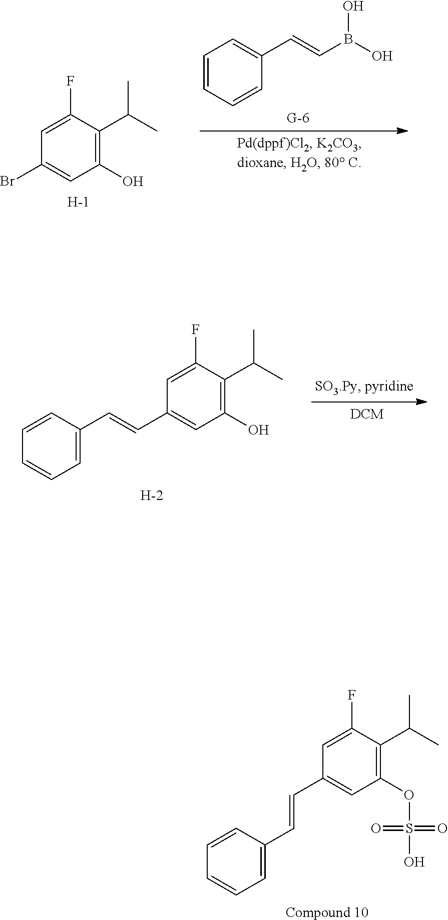

To a solution of 5-bromo-3-fluoro-2-isopropylphenol (360.0 mg, 1.54 mmol) in 1,4-dioxane/H₂O (10 mL/1 mL) was added [(E)-2-phenylethenyl]boranediol (228.5 mg, 1.54 mmol), Pd(dppf)Cl₂ (113.0 mg, 0.15 mmol) and K₂CO₃ (426.9 mg, 3.09 mmol). The reaction mixture was stirred at 80° C. for 12 h under nitrogen. The desired mass was detected on LC-MS. The solvent was removed by vacuum. The residue was quenched with water, extracted with EtOAc, the combined organic phase was washed with brine, dried over anhydrous Na₂SO₄, filtered and concentrated. The residue was purified by silica gel chromatography (eluting with PE/EtOAc from 100/0 to 40/60 in 30 mins) and C₁₈ column (eluting with 0.5% NH₃·H₂O/MeCN from 100:0 to 30:70 in 30 mins) to afford H-2. LCMS (ESI): calcd for C₁₇H₁₇FO [M−H]⁺ ms/z 255.1, found 254.9. ¹H NMR (400 MHz, DMSO-d₆, ppm) 9.79 (s, 1H), 7.59 (d, J=7.6 Hz, 2H), 7.37 (t, J=7.6 Hz, 2H), 7.27 (t, J=7.6 Hz, 1H), 7.10 (s, 2H), 6.86 (d, J=12.4 Hz, 1H), 6.79 (s, 1H), 3.39-3.34 (m, 1H), 1.25 (d, J=7.2 Hz, 6H).

To a solution of H-2 (100.0 mg, 0.39 mmol) in DCM (5 ml) were added SO₃·Py (310.55 mg, 1.95 mmol) and pyridine (462.15 mg, 5.85 mmol). The reaction mixture was stirred at 25° C. for 6 hours. The desired mass was detected on LC-MS. The solvent was removed under reduce pressure. The residue was purified by C₁₈ column (eluting with ACN-H₂O (0.1% HCOOH) from 0:100 to 50:50 in 30.0 min) to afford compound 10. LCMS (ESI): calcd for C₁₇H₁₇FO₄S [M−H]⁻ m/z 335.0, found 335.2. ¹H NMR (400 MHz, CD₃OD, ppm) 7.54 (d, J=7.6 Hz, 2H), 7.49 (s, 1H), 7.33 (t, J=7.6 Hz, 2H), 7.23 (t, J=7.2 Hz, 1H), 7.16 (d, J=16.4 Hz, 1H), 7.11-7.03 (m, 2H), 3.58 (hept, J=7.2 Hz, 1H), 1.32 (d, J=7.2 Hz, 6H).

### Preparation of 3-fluoro-2-isopropyl-5-(4-methylquinolin-7-yl)phenyl dihydrogen phosphate (Compound 11)

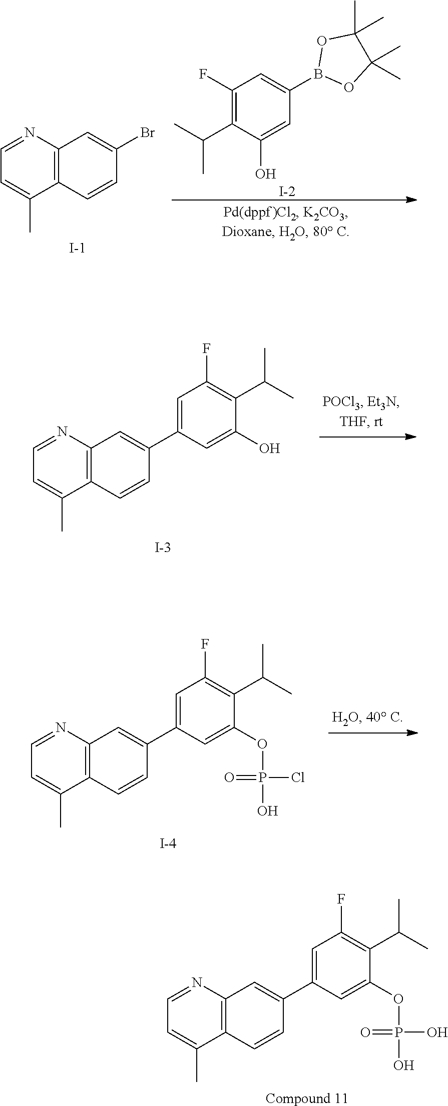

To a solution of 7-bromo-4-methylquinoline (100.0 mg, 0.45 mmol) in 1,4-dioxane/H₂O=5:1 (3 mL) was added 3-fluoro-2-isopropyl-5-(4,4,5,5-tetramethyl-1,3,2-dioxaborolan-2-yl)phenol (151.4 mg, 0.54 mmol), Pd(dppf)Cl₂ (36.6 mg, 0.05 mmol) and Potassium carbonate (124.2 mg, 0.90 mmol). The reaction was stirred for 12 h at 80° C. under N₂. The desired mass was detected on LC-MS. The solvent was removed under reduce pressure. Then the mixture quenched with Water (10 mL), extracted with EtOAc. The combined organic layers are washed with brine, dried over anhydrous Na₂SO₄, filtered, and concentrated under vacuum. The residue was purified by flash chromatography (eluting with PE/EtOAc from 100/0 to 60/40 in 30 mins) and concentrated under vacuum. The crude product was triturated with diethyl ether to afford 1.3. LCMS (ESI): calcd for C₁₉H₁₉FNO [M+H]⁺ m/z 296.3, found 296.2. ¹H NMR (400 MHz, DMSO-d₆, ppm) 10.00 (s, 1H), 8.78 (d, J=4.4 Hz, 1H), 8.18-8.13 (m, 2H), 7.87-7.85 (m, 1H), 7.39 (d, J=4.0 Hz, 1H), 7.13-7.00 (m, 2H), 3.46-3.39 (m, 1H), 2.71 (s, 3H), 1.30 (d, J=7.2 Hz, 6H).

To a solution of I-3 (430.0 mg, 1.45 mmol) in THF (6 mL) were added POCl₃ (733.7 mg, 4.79 mmol) and Et₃N (880.4 mg, 8.70 mmol) dropwise. The reaction was stirred for 6 h at 25° C. The desired mass was detected on LC-MS. The solvent was removed under reduce pressure to afford compound 1-4, which was used directly for the next step. LCMS (ESI): calcd for C₁₉H₁₃ClFNO₃P [M+H]⁺ m/z 394.0, found 393.9.

To a solution of 3-fluoro-2-isopropyl-5-(4-methylquinolin-7-yl)phenyl hydrogen phosphorochloridate (400.0 mg, 1.02 mmol) in H₂O (6 mL). The reaction was stirred for 6 h at 40° C. The desired mass was detected on LC-MS. The residue was purified by prep-HPLC (columns: Gemini 5 um C18 150×21.2 mm, mobile phase: H₂O-ACN (0.1% TFA), gradient: 90-50, 10.0 min) to afford compound 11. LCMS (ESI): calcd for C₁₉H₁₉FNO₄P [M+H]⁺ m/z 376.3, found 376.2. ¹H NMR (400 MHz, DMSO-d₆, ppm): 8.89 (s, 1H), 8.35 (s, 1H), 8.26 (d, J=8.8 Hz, 1H), 8.00 (d, J=8.8 Hz, 1H), 7.65 (s, 1H), 7.57-7.46 (m, 2H), 3.51-3.44 (m, 1H), 2.78 (s, 3H), 1.32 (d, J=7.2 Hz, 6H).

### Caco-2 and S9 Stability Studies

Compounds were tested under typical Caco-2 and S9 assay conditions. The results are shown in Table A.

TABLE A

| | | Caco-2, AB | mouse S9 Clᵢₙₜ |
|---|---|---|---|
| | Structure | (10⁻⁶ cm/s) | uL/min/mg |
| 1 | 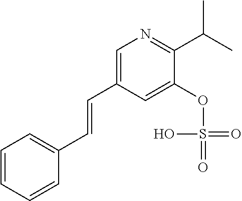 [SMILES: CC(C)c1ncc(/C=C/c2ccccc2)cc1OS(=O)(=O)O] | 0.44 | 4.91 |
| 2 | 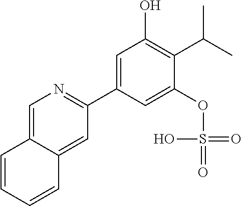 [SMILES: CC(C)c1c(O)cc(-c2cc3ccccc3cn2)cc1OS(=O)(=O)O] | | 1.18 |
| 3 | 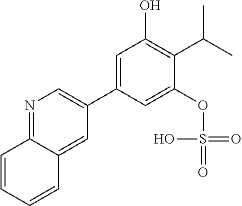 [SMILES: CC(C)c1c(O)cc(-c2cnc3ccccc3c2)cc1OS(=O)(=O)O] | 0.29 | 0.00 |
| 4 | 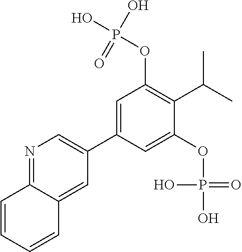 [SMILES: CC(C)c1c(OP(=O)(O)O)cc(-c2cnc3ccccc3c2)cc1OP(=O)(O)O] | <0.20 | 3.43 |
| 5 | 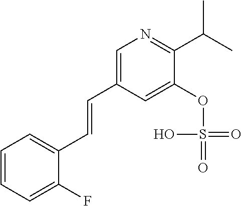 [SMILES: CC(C)c1ncc(/C=C/c2ccccc2F)cc1OS(=O)(=O)O] | 0.44 | 2.64 |
| 6 | 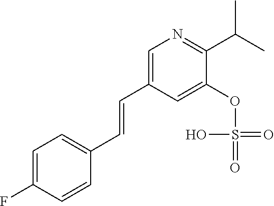 [SMILES: CC(C)c1ncc(/C=C/c2ccc(F)cc2)cc1OS(=O)(=O)O] | 0.62 | 1.26 |
| 7 | 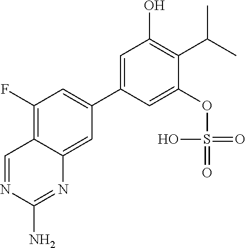 [SMILES: CC(C)c1c(O)cc(-c2cc(F)c3cnc(N)nc3c2)cc1OS(=O)(=O)O] | 0.37 | |
| 8 | 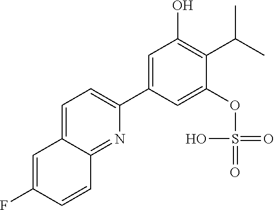 [SMILES: CC(C)c1c(O)cc(-c2ccc3cc(F)ccc3n2)cc1OS(=O)(=O)O] | | 0.02 |
| 9 | 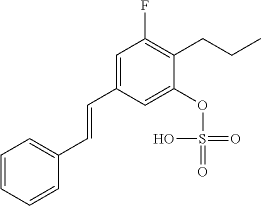 [SMILES: CCCc1c(F)cc(/C=C/c2ccccc2)cc1OS(=O)(=O)O] | 0.29 | 0.70 |
| 10 | 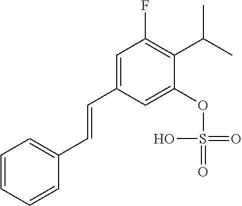 [SMILES: CC(C)c1c(F)cc(/C=C/c2ccccc2)cc1OS(=O)(=O)O] | 0.25 | 0.47 |
| 11 | 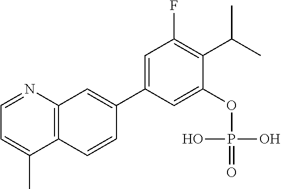 [SMILES: Cc1ccnc2cc(-c3cc(F)c(C(C)C)c(OP(=O)(O)O)c3)ccc12] | | 0.68 |

### Ex Vivo Release Studies

The ability of the compounds of Formula (I) to release the active compound in the small or large intestine is assessed by introducing representative compounds of the disclosure to the contents of either a rodent small intestine or a rodent large intestine for up to 24 hours at 37° C. The amount of active drug released after the incubation period is determined by LC-MS/MS. Additional studies are conducted by incubating representative compounds of the disclosure with human fecal samples.

In Vitro Anaerobic Metabolism in Fecal Homogenates (vs. Control)

| | | | Metabolic | |
|---|---|---|---|---|
| | Test | | Incubation | |
| | Conc | | Time-Course | |
| Study Design | (μM) | Animal No. | (hr) | LC-MS/MS Assay |
| Compound 1 Stability in | 20 μM | 6M + 6F, N = 3 | 0, 1, 2, | LC-MS/MS assay |
| Fecal homogenate | | replicates | 4, 6 and 24 | for disappearance |
| | | | | of Compound 1 and |
| Compound 1 Stability in BHI | 20 μM | N = 3 | 0, and 24 | the formation of its |
| Broth (Vehicle Stability | | replicates | | metabolite (the |
| control) | | | | active agent) |

The structure of Compound 1 is

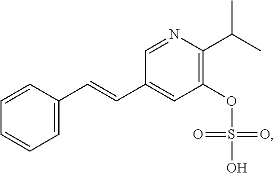 [SMILES: CC(C)c1ncc(/C=C/c2ccccc2)cc1OS(=O)(=O)O]

and its metabolite (as the active AHR agent) is

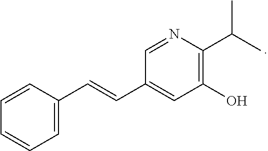 [SMILES: CC(C)c1ncc(/C=C/c2ccccc2)cc1O]

The AHR activity of the metabolite is measured as AHR reEC₅₀ (as % activation to tapinarof at 1 μM)—of 166.1 nM (69.5%) and a AHR abEC₅₀ of 241.5 nM.

Compound 1 was dissolved in PBS (pH 7.4) to provide a 200 μM working solution. An aliquot of 50 μL volume was spiked to each 450 μL fecal homogenate incubation sample to provide an incubation concentration at 20 μM.

An estimated 1 g to 2 g wet weight of fecal material from healthy mice and rats (separately) was immediately collected in a pre-labelled 50 mL EP tube and stored at ambient room temperature. Based on the weight of each fecal material, a dilution with 0.01 M isotonic phosphate buffer (pH 7.0) is performed to provide a 12.5× diluted fecal homogenate. All fecal materials were stored at room temperature before being used in fecal homogenate preparation.

Under anaerobic conditions, each of the fecal specimen materials collected in 0.01 M isotonic phosphate buffer (pH 7.0) was homogenized by shaking and mixing with a stirrer. Each lot of the fecal homogenate material was added with an appropriate volume of 0.01 M isotonic phosphate buffer (pH 7.0) to provide fecal homogenate at 12.5× dilution. Under anaerobic condition in a nitrogen glove box purged with high purity nitrogen to ensure <1% oxygen level, the fecal homogenate was transferred to a clean container and diluted with 3 volumes of Brain Heart Infusion Broth (BHI) to provide a 50× diluted fecal homogenate. The diluted fecal homogenate matrix at an aliquot of 0.45 mL was dispensed into 2-mL EP tube, freshly prepared Compound 1 working solutions were then spiked to the fecal homogenate sample vials for incubation over the metabolic stability time-course.

Compound 1 was incubated at a target concentration of 20 μM in each fecal homogenate over a time course of 0, 1, 2, 4, 6 and 24 hours. Metabolic incubation at each time point was performed in triplicate. Incubation of the samples was performed in 2-mL EP tube. Into each pre-labeled sample vial, 50 μL of a Compound 1 working solution was accurately dispensed followed by the addition of the 0.45 mL pre-incubated fecal homogenate. Sample were capped, vortex mixed and incubated under an anaerobic environment at 37° C. At the end of each incubation time point, 1.0 mL of IS (Internal Standard) as a stop solvent was added to each sample with vortex mixed to stop the metabolic reactions. Samples at the 0-hour time point were pre-treated with the addition of IS stop solvent prior to the spiking of fecal homogenate and Compound 1. All samples were stored at −80° C. prior to LC-MS/MS assay. Samples are analyzed by LC-MS/MS. The MS detection is performed using a Sciex API 5500+ instrument. Each compound is analyzed by reverse phase HPLC.

The results are shown in the below table.

### Compound 1 Conversion to Metabolite Overtime in Rat or Mouse Fecal Homogenate Compared to Control

| | Compound 1 Conc. (nM) | | Compound 1 Metabolite (nM) | |
|---|---|---|---|---|
| Time (hr) | Average | STD | Average | STD |

Mouse fecal homogenate

| | | | | |
|---|---|---|---|---|
| 0.0 | 23700 | 283 | 171 | 4.24 |
| 1 | 21800 | 424 | 1006 | 19.8 |
| 2 | 21600 | 990 | 1725 | 7.07 |
| 4 | 19200 | 849 | 3375 | 106 |
| 6 | 14750 | 70.7 | 5690 | 70.7 |
| 24 | 73.8 | 16.9 | 13750 | 354 |

Rat fecal homogenate

| | | | | |
|---|---|---|---|---|
| 0.0 | 25050 | 1626 | 167 | 7.07 |
| 1 | 21300 | 141 | 1435 | 21.2 |
| 2 | 20550 | 495 | 3235 | 134 |
| 4 | 14200 | 0 | 6880 | 70.7 |
| 6 | 3020 | 84.9 | 13550 | 495 |
| 24 | 65.7 | 8.49 | 14850 | 778 |

BHI

| | | | | |
|---|---|---|---|---|
| 0.0 | 23200 | 3111 | 157 | 14.8 |
| 24 | 21900 | 566 | 126 | 12.7 |

It should be appreciated that all combinations of the foregoing concepts and implementations and additional concepts and implementations discussed in greater detail below are contemplated as being part of the inventive subject matter disclosed herein, and may be employed in any suitable combination to achieve the benefits as described herein. In particular, all combinations of claimed subject matter appearing at the end of this disclosure are contemplated as being part of the inventive subject matter disclosed herein

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular cases, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A compound having a structure of Formula (I): 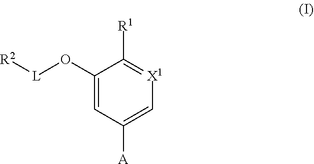
wherein:
A is —Y¹—Z¹ or a fused bicyclic heteroaryl group having 8-10 total ring atoms and 1, 2, or 3 heteroatoms selected from N, O, or S, and the heteroaryl is optionally substituted with 1, 2, or 3 R^{a};
Y¹ is 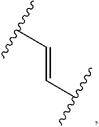 [SMILES: */C=C/*]
O, S, SO, or SO₂;
Z¹ is phenyl or a 5- to 6-membered heteroaryl having 1, 2, or 3 heteroatoms selected from N, O, and S, and the phenyl or heteroaryl is optionally substituted with 1, 2, or 3 R^{a};
X¹ is N, CR³ or C—O-L-R²;
R¹ is C₁₋₄₅alkyl, C₁₋₅haloalkyl, C₁₋₅hydroxyalkyl, C₃₋₅cycloalkyl, OC₁₋₅alkyl, N(R^{N})₂, C(═O)C₁₋₄ alkyl, or C(═CH)C₁₋₄alkyl;
each R² independently is SO₂—R⁴, PO(R⁴)₂, CO₂H, N(R^{N})₂, N(H)—C₂₋₆alkylene-N(R^{N})₂, N(H)—C₂₋₆polyoxyalkylene-N(R^{N})₂, NHCH(R⁵)CO₂H, NH—C₁₋₅alkylene-CH(NH₂)CO₂H, a saccharide, or NH-phenyl wherein the phenyl is optionally substituted with 1, 2, or 3 R^{b};
R³ is halo, C₁₋₆alkoxy, OH, or N(R^{N})₂;
each L independently is C(O), C(O)—Wₙ—C(O), C(O)—Wₙ, or C(O)NH—Wₙ, or when R² is SO₂—R⁴, PO(R⁴)₂, or a saccharide, L can be a bond;
each W independently is CH₂, C₂₋₃alkylene-O, or O—C₂₋₃alkylene;
n is 1, 2, 3, 4, 5, or 6;
each R⁴ independently is OH or NH₂;
each R⁵ independently is H, C₁₋₅alkyl, or C₁₋₅alkylene-R⁶;
each R⁶ independently is CO₂H, CO₂C₁₋₃alkyl, CON(R^{N})₂, OH, OCH₃, N(R^{N})₂, NHC(NH)NH₂, heteroaryl comprising 5 or 6 total ring atoms and 1, 2, or 3 heteroatoms selected from N, O, and S, or phenyl, wherein the heteroaryl or phenyl is optionally substituted with 1, 2, 3, 4, or 5 C₀₋₂alkylene-R^{b} substituents;
each R^{a} independently is halo, C₁₋₅alkyl, CO₂H, CO₂C₁₋₃alkyl, CON(R^{N})₂, OH, OC₁₋₃alkyl, or N(R^{N})₂;
each R^{b} independently is CO₂H, CO₂C₁₋₃alkyl, or OH; and
each R^{N} independently is H or C₁₋₃alkyl;
or a pharmaceutically acceptable salt thereof,
provided that when Y¹ is 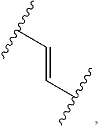 [SMILES: */C=C/*]
Z¹ is unsubstituted phenyl, and R¹ is isopropyl, then X¹ is N or CR³, and R³ is halo, C₁₋₆alkoxy, or N(R^{N})₂.

2. The compound or salt of claim 1, wherein A is Y¹—Z¹.

3. The compound or salt of claim 2, wherein Y¹ is

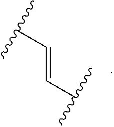 [SMILES: */C=C/*]

4. The compound or salt of claim 2, wherein Y¹ is S, SO, or SO₂.

5. The compound or salt of any of claims 2 to 4, wherein Z¹ is phenyl.

6. The compound or salt of any one of claims 2 to 4, wherein Z¹ is a 5- to 6-membered heteroaryl.

7. The compound or salt of claim 6, wherein Z¹ is pyridine, pyrazole, imidazole, or pyrimidine.

8. The compound or salt of any one of claims 5 to 7, wherein Z¹ is unsubstituted.

9. The compound or salt of claims 5 to 7, wherein Z¹ is substituted with 1 or 2 R^{a} groups.

10. The compound or salt of claim 9, wherein each R^{a} is independently halo, C₁₋₃alkyl, or NH₂.

11. The compound or salt of claim 1, wherein A is a fused bicyclic heteroaryl.

12. The compound or salt of claim 11, wherein A is a 10-membered heteroaryl.

13. The compound or salt of claim 11 or 12, wherein A is isoquinoline, quinoline, or quinazoline.

14. The compound or salt of claim 13, wherein A is 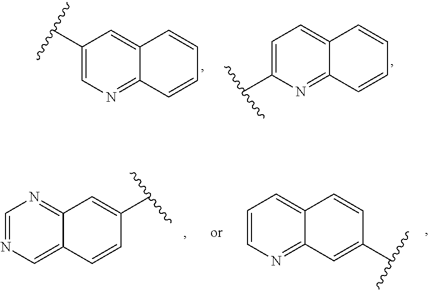 [SMILES: *c1cnc2ccccc2c1 ; *c1ccc2ccccc2n1 ; *c1ccc2cncnc2c1 ; *c1ccc2cccnc2c1]
or
when R² is SO₂—R⁴ or PO(R⁴)₂, A can be

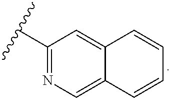 [SMILES: *c1cc2ccccc2cn1]

15. The compound or salt of any one of claims 11 to 14, wherein A is unsubstituted.

16. The compound or salt of any one of claims 11 to 14, wherein A is substituted with 1 or 2 R^{a} groups.

17. The compound or salt of claim 16, wherein each R^{a} is independently halo, C₁₋₃alkyl, or NH₂.

18. The compound or salt of any one of claims 1 to 17, wherein X¹ is N.

19. The compound or salt of any one of claims 1 to 17, wherein X¹ is CR³.

20. The compound or salt of claim 19, wherein R³ is F or OH.

21. The compound or salt of any one of claims 1 to 17, wherein X¹ is C—O-L-R².

22. The compound or salt of claim 21, wherein X¹ is C—O—R².

23. The compound or salt of claim 22, wherein X¹ is C—OSO₂—R⁴ or C—OPO(R⁴)₂.

24. The compound of or salt of any one of claims 1 to 23, wherein R¹ is C₁₋₅alkyl, C₁₋₅ haloalkyl, or C₁₋₅hydroxyalkyl.

25. The compound or salt of claim 24, wherein R¹ is C₁₋₅alkyl.

26. The compound or salt of claim 25, wherein R¹ is C₃alkyl.

27. The compound or salt of any one of claims 1 to 23, wherein R¹ is OC₁₋₅alkyl, N(R^{N})₂, or C(═O)C₁₋₄alkyl.

28. The compound or salt of any one of claims 1 to 27, wherein L is a bond.

29. The compound or salt of any one of claims 1 to 27, wherein L is C(O), C(O)—Wₙ—C(O), C(O)—Wₙ, or C(O)NH—Wₙ.

30. The compound or salt of claim 29, wherein each W is independently CH₂ or C₂₋₃alkylene-O.

31. The compound or salt of claim 29 or 30, wherein n is 1 or 2.

32. The compound of or salt of any one of claims 1 to 31, wherein R² is SO₂—R⁴ or PO(R⁴)₂.

33. The compound or salt of claim 32, wherein R² is SO₂H or PO(OH)₂.

34. The compound or salt of any one of claims 1 to 31, wherein R² is N(H)—C₂₋₆alkylene-N(R^{N})₂, N(H)—C₂₋₆polyoxyalkylene-N(R^{N})₂, NHCH(R⁵)CO₂H, or NH—C₁₋₆alkylene-CH(NH₂)CO₂H.

35. The compound or salt of claim 34, wherein R² is N(H)—C₂₋₆alkylene-N(R^{N})₂ or NH—C₁₋₆ alkylene-CH(NH₂)CO₂H.

36. The compound or salt of claim 35, wherein R² is NHCH(R⁵)CO₂H.

37. The compound or salt of claim 36, wherein R⁵ is C₁₋₆alkyl or C₁₋₆alkylene-R⁶.

38. The compound or salt of claim 37, wherein R⁶ is CO₂H, CO₂CH₃, CONH₂, OH, OCH₃, NH₂, NHC(NH)NH₂, imidazolyl, phenyl, or 4-hydroxy-phenyl.

39. The compound or salt of any one of claims 1 to 31, wherein at least one R² is NH-phenyl.

40. The compound or salt of claim 39, wherein the phenyl is substituted with 1 or 2 R^{b} groups.

41. The compound or salt of any one of claims 1 to 40, wherein each R^{N} is independently H or CH₃.

42. The compound or salt of claim 1 selected from the group consisting of:
(E)-2-isopropyl-5-styrylpyridin-3-yl hydrogen sulfate,
(E)-2-isopropyl-5-styrylpyridin-3-yl dihydrogen phosphate,
3-hydroxy-2-isopropyl-5-(isoquinolin-3-yl)phenyl hydrogen sulfate,
3-hydroxy-2-isopropyl-5-(isoquinolin-3-yl)phenyl dihydrogen phosphate,
3-hydroxy-2-isopropyl-5-(quinolin-3-yl)phenyl hydrogen sulfate,
3-hydroxy-2-isopropyl-5-(quinolin-3-yl)phenyl dihydrogen phosphate,
2-isopropyl-5-(quinolin-3-yl)-1,3-phenylene bis(hydrogen sulfate),
2-isopropyl-5-(quinolin-3-yl)-1,3-phenylene bis(dihydrogen phosphate),
(E)-5-(2-fluorostyryl)-2-isopropylpyridin-3-yl hydrogen sulfate,
(E)-5-(2-fluorostyryl)-2-isopropylpyridin-3-yl dihydrogen phosphate,
(E)-5-(3-fluorostyryl)-2-isopropylpyridin-3-yl hydrogen sulfate,
(E)-5-(3-fluorostyryl)-2-isopropylpyridin-3-yl dihydrogen phosphate,
(E)-5-(4-fluorostyryl)-2-isopropylpyridin-3-yl hydrogen sulfate,
(E)-5-(4-fluorostyryl)-2-isopropylpyridin-3-yl dihydrogen phosphate,
5-(2-amino-5-fluoroquinazolin-7-yl)-3-hydroxy-2-isopropylphenyl hydrogen sulfate,
5-(2-amino-5-fluoroquinazolin-7-yl)-3-hydroxy-2-isopropylphenyl dihydrogen phosphate,
5-(2-amino-5-fluoroquinazolin-7-yl)-2-isopropyl-1,3-phenylene bis(hydrogen sulfate);
5-(2-amino-5-fluoroquinazolin-7-yl)-2-isopropyl-1,3-phenylene bis(dihydrogen phosphate);
5-(6-fluoroquinolin-2-yl)-3-hydroxy-2-isopropylphenyl hydrogen sulfate,
5-(6-fluoroquinolin-2-yl)-3-hydroxy-2-isopropylphenyl dihydrogen phosphate,
5-(6-fluoroquinolin-2-yl)-2-isopropyl-1,3-phenylene bis(hydrogen sulfate),
5-(6-fluoroquinolin-2-yl)-2-isopropyl-1,3-phenylene bis(dihydrogen phosphate),
(E)-3-fluoro-2-propyl-5-styrylphenyl hydrogen sulfate,
(E)-3-fluoro-2-propyl-5-styrylphenyl dihydrogen phosphate,
(E)-3-fluoro-2-isopropyl-5-styrylphenyl hydrogen sulfate,
(E)-3-fluoro-2-isopropyl-5-styrylphenyl dihydrogen phsophate,
3-fluoro-2-isopropyl-5-(4-methylquinolin-7-yl)phenyl hydrogen sulfate, and
3-fluoro-2-isopropyl-5-(4-methylquinolin-7-yl)phenyl dihydrogen phosphate.

43. The compound or salt of claim 42 selected from the group consisting of: 2-isopropyl-5-(quinolin-3-yl)-1,3-phenylene bis(dihydrogen phosphate), 2-isopropyl-5-(quinolin-3-yl)-1,3-phenylene bis(hydrogen sulfate), (E)-5-(2-fluorostyryl)-2-isopropylpyridin-3-yl hydrogen sulfate, (E)-5-(2-fluorostyryl)-2-isopropylpyridin-3-yl dihydrogen phosphate, (E)-3-fluoro-2-propyl-5-styrylphenyl hydrogen sulfate, (E)-3-fluoro-2-propyl-5-styrylphenyl dihydrogen phosphate, (E)-3-fluoro-2-isopropyl-5-styrylphenyl hydrogen sulfate, (E)-3-fluoro-2-isopropyl-5-styrylphenyl dihydrogen phosphate, 3-fluoro-2-isopropyl-5-(4-methylquinolin-7-yl)phenyl hydrogen sulfate, and 3-fluoro-2-isopropyl-5-(4-methylquinolin-7-yl)phenyl dihydrogen phosphate.

44. A pharmaceutical formulation comprising the compound or salt of any one of claims 1 to 43 and a pharmaceutically acceptable excipient.

45. A method of modulating the aryl hydrocarbon receptor (AHR) activity in a subject, comprising contacting the AHR with the compound or salt of any one of claims 1 to 43 in an amount effective to modulate the AHR activity.

46. A method of treating or preventing a disease or disorder associated with aberrant aryl hydrocarbon receptor (AHR) activity in a subject, comprising administering to the subject a therapeutically effective amount of the compound or salt of any one of claims 1 to 43.

47. The method of embodiment 46, wherein the administration is topical.

48. The method of embodiment 46, wherein the administration is oral or via injection or infusion.

49. The method of claim 46, 47, or 48, wherein the disease or disorder is an inflammatory disease or disorder.

50. The method of claim 46, 47, or 48, wherein the disease or disorder is a disease or disorder of the gastrointestinal tract, skin, lung, central nervous system, pancreas, eye, bones, bone joints, a neuroinflammatory disease, or a neurodegenerative disease.

51. The method of claim 50, wherein the disease or disorder of the gastrointestinal tract is selected from the group consisting of colitis, inflammatory bowel disease, Crohn's disease, celiac disease, necrotizing enterocolitis, irritable bowel syndrome, chronic idiopathic constipation, traveler's diarrhea, and colorectal cancer.

52. The method of claim 50, wherein the disease or disorder of the skin is selected from the group consisting of atopic dermatitis, acne, psoriasis, and vitiligo.

53. The method of claim 50, wherein the disease or disorder of the eye is abnormal eye movements, inflammatory ocular disease, autoimmune ocular disease, hereditary ocular disease, degenerative ocular disease, vascularization ocular disease, dry and wet age-related macular degeneration (“AMD”), Uveitis, retinitis pigmentosa (“RP”), primary open-angle glaucoma (“POAG”), primary congenital glaucoma, Behcet's disease, or Leber congenital amaurosis (“LCA”).

54. The method of claim 50, wherein the disease or disorder of the lung is lung fibrosis, asthma or chronic obstructive pulmonary disease.

55. The method of claim 50, wherein the disease or disorder of the bone joints is osteoporosis, rheumatoid arthritis, or bone cancer.

56. The method of claim 46, 47, or 48, wherein the disease or disorder is diabetes, cancer, a viral infection, or a bacterial infection.

57. The method of claim 56, wherein the viral infection is flavivirus infection or coronavirus infection.

58. The method of claim 56, wherein the bacterial infection is a pulmonary infection, gastrointestinal infection, skin infection, ear infection or a septicemia.

59. The method of any one of claims 46 to 58, further comprising administration of a therapeutic agent.

60. The method of claim 59, wherein the therapeutic agent is an anti-inflammatory agent.

61. The method of claim 60, wherein the anti-inflammatory agent is selected from mesalazine, naproxen, ibuprofen, diclofenac, celecoxib, sulindac, oxaprozin, piroxicam, indomethacin, meloxicam, fenoprofen, difunisal, etodolac, ketorolac tromethamine, meclofenamate, nabumetone, salsalate, or any combination of the foregoing.
